(19) United States
(12) Patent Application Publication
(10) Pub. No.: US 2023/0414274 A1
(43) Pub. Date: Dec. 28, 2023

(54) NANOSECOND PULSING TO TREAT A BODY LUMEN

(71) Applicant: PULSE BIOSCIENCES, INC., Hayward, CA (US)

(72) Inventors: Kevin L. MOSS, Lathrop, CA (US); Richard J. CONNOLLY, Riverview, FL (US); David J. DANITZ, San Jose, CA (US); Mitchell E. LEVINSON, Pleasanton, CA (US); Darrin R. UECKER, San Mateo, CA (US); Cameron D. HINMAN, Thurmond, NC (US)

(21) Appl. No.: 18/252,812

(22) PCT Filed: Nov. 12, 2021
(86) PCT No.: PCT/US2021/059269

§ 371 Date: May 12, 2023

Related U.S. Application Data
(60) Provisional application No. 63/113,804, filed on Nov. 13, 2020.
(60) Provisional application No. 63/236,186, filed on Aug. 23, 2021.

(51) Int. Cl. A61B 18/14

(52) U.S. Cl. CPC A61B 18/1492; A61B 2018/0016

(57) ABSTRACT

Described herein are elongate applicator tools adapted to be inserted into an anatomical structure of a body to deliver electric treatment, for example, high voltage, sub-microsecond electrical energy, to a target tissue. These applicator tools may be configured for insertion into a blood vessel, an otolaryngological structure (such as an ear, nose, or throat, including anatomical structures, such as a turbinate, tonsils, tongue, soft palate, parotid glands, esophageal glands), a gastrointestinal tract (e.g., stomach, small intestine, large intestine, duodenum, colon, etc., including the esophagus), structures of the female reproductive system (e.g., fallopian tubes, uterus), and/or a respiratory tract (e.g., trachea, pharynx, larynx, and bronchi). Also disclosed herein are systems including these tools and methods of their operation.

### CROSS REFERENCE TO RELATED APPLICATIONS

This patent application claims priority to U.S. Provisional Patent Application No. 63/113,804, tiled “NANOSECOND PULSING TO TREAT A BODY LUMEN,” filed on Nov. 13, 2020, and U.S. Provisional Patent Application No. 63/236,186, titled “NANOSECOND PULSING TO TREAT A BODY LUMEN,” filed on Aug. 23, 2021, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are medical apparatuses (e.g., devices, systems, etc.) and methods that may be used to perform medical operations or procedures to treat patients. Specifically, the apparatuses described herein can include tubular treatment tips that are configured for use within various anatomical structures such as, but not limited to body lumen, including without limitation an otolaryngological lumen or structure (e.g., the nose, ear, throat, tongue, etc.), as well as any part of esophagus, duodenum, small or large intestine and stomach.

### BACKGROUND

Short, high-field strength electric pulses have been described for electromanipulation of biological cells. For example, electric pulses may be used in treatment of human cells and tissue including tumor cells, such as basal cell carcinoma, squamous cell carcinoma, and melanoma. The voltage induced across a cell membrane may depend on the pulse length and pulse amplitude. Pulses longer than about 1 microsecond may charge the outer cell membrane and may lead to permanent opening of pores. Permanent openings may result in instant or near instant cell death. Pulses shorter than about 1 microsecond may affect the cell interior without adversely or permanently affecting the outer cell membrane and result in a delayed cell death with intact cell membranes. Such shorter pulses with a field strength varying in the range, for example, of 10 kV/cm to 100 kV/cm may trigger apoptosis (i.e. programmed cell death) in some or all of the cells exposed to the described field strength and pulse duration. These higher electric field strengths and shorter electric pulses may be useful in manipulating intracellular structures, such as nuclei and mitochondria. For example, such sub-microsecond (e.g., nanosecond) high voltage pulse generators have been proposed for biological and medical applications.

It would be particularly advantageous to be able to treat a patient within various anatomical structures, for example, one or more lumen of the ear, nose, throat, or esophagus. However, because of the high therapeutic voltages and due to the risk of damaging tissues or otherwise harming the patient no known devices and instruments using such high voltage energy modalities have been developed. The risks of delivering high voltage energy, such risks including electrical shock, arcing, burns, internal-organ damage, and cardiac arrhythmias, are even more acute when the high voltage device is intended to be inserted into the body.

Thus, it would be beneficial to provide devices, such as applicators, including elongate applicators, and corresponding methods that may apply high voltage, sub-microsecond electrical pulses to treat patients while mitigating the above-mentioned risks.

### SUMMARY OF THE DISCLOSURE

Described herein are apparatuses and methods for treating within a body lumen using electric fields, including sub-microsecond (e.g., nanosecond) pulsed electrical fields. In particular, the methods and apparatuses described herein may be configured to selectively treat, for example, a portion of a wall of a body lumen, e.g., the outermost layer(s), with nanosecond pulsed electrical fields that may prevent damage to deeper, non-target regions.

These methods and apparatuses may provide non-thermal treatment of tissue, including non-thermal ablation of cells using pulsed, sub-microsecond energy applied by the apparatuses described herein. Also described herein are methods and apparatuses for applying non-thermal, pulsed, sub-microsecond electrical fields to selectively target tissue on or in a body lumen without substantially effecting non-target tissues. Thus, in some implementations these methods and apparatuses have particular advantages over thermal apparatuses and methods, including in particular the application of radio frequency (RF) energy to ablate tissue within a lumen. In particular, these methods and apparatuses may be configured to operate with non-thermal, pulsed, sub-microsecond electrical energy.

In some examples, the instruments and methods described herein are configured for otolaryngological use, e.g., for insertion and treatment by applying nanosecond pulsed electrical fields within a lumen or other otolaryngological structure, such as an ear, nose, or throat, including anatomical structures, such as turbinates, tonsils, tongue, soft palate, parotid glands, as well as those structures that connect throat (pharynx) to the stomach. These instruments and devices may be configured for insertion into these structures. For example, they may be configured as an elongate applicator tools, applicator catheters, tube, etc. sized and shaped to fit within an ear, nose, throat, and/or to treat an associated anatomical structure or passageway (e.g., turbinates, tonsils, tongue, soft palate, parotid gland, esophageal and other submucosal glandular structures, etc.). For example, these methods and apparatuses may apply nanosecond pulsed electrical fields to treat a mucosal epithelium, nasal polyps, mucous membrane papillomas, vocal fold carcinomas, and/or any other head and neck disease, lesion or tumor.

Also described herein are methods and apparatuses configured for the delivery of electric treatment (e.g., nanosecond pulsed electrical fields) to blood vessels, structures of the female reproductive system (e.g., fallopian tubes, uterus), or a portion of the gastrointestinal tract (e.g., stomach, small intestine, large intestine, duodenum, colon, etc., including, but not limited to the esophagus).

Also described herein are methods and apparatuses configured for the delivery of nanosecond pulsed electrical fields to a portion of the respiratory tract, including the trachea, pharynx, larynx, and bronchi. For convenience of the description, all anatomical structures, passageways, cavities, tubes, lumens or vessels that can be treated using apparatuses and methods of the present disclosure will each be referred to generally as a body lumen or a lumen or, in some examples, it may be referred to some specific anatomical structures, such as esophagus, submucosal glandular structures, etc.

In general, these methods may include treatment of a subject (e.g., patient) by the application of therapeutic energy, including but not limited to short, high field strength electric pulses, while minimizing or avoiding the risk of harming non-target tissue. In some examples, these applicators may be referred to herein as applicator tools, and may be used for minimally invasive procedures, and may be particularly well suited, for example, for treatments of various conditions, disorders and diseases, such as, but not limited to cancer (and other types of abnormal tissue growth), and the like. These applications may be also particularly well suited for use with various fully and partially automated systems, such as robotic systems. In particular, the apparatuses described herein may be configured as apparatuses (e.g., catheter apparatuses, catheter applicator tips, etc.) that can be used with a variety of different generator systems, as will be described in greater detail herein. Thus, the methods and apparatuses (e.g., systems and/or devices) described herein may be used to treat any anatomical structure (e.g., a vein, artery, vessel, heart, trachea, pharynx, larynx, bronchi, ureter, urethra, fallopian tubes, cervix, uterus, intestine (large and/or small), pancreas and pancreatic duct, liver and hepatic ducts, rectum, esophagus, stomach, nasal cavity, seminal vesicles, bronchi, submucosal glands, etc.). In particular, these methods and apparatuses may be used to treat cancers, such as, but not limited to pancreatic cancer, lung cancer, etc.

Thus, the apparatuses described herein may be configured for manual or automated (e.g., robotic-assisted) control. In some examples these apparatuses may be integrated into systems that are configured to be mounted onto or coupled to a movable (e.g., robotic) arm of a robotic system, such as robotic medical treatment system or robotic surgical system. For convenience of description the present disclosure may refer to these as robotic systems, however, it should be understood that such robotic systems are intended to cover any robotic medical treatment system (including surgical and non-surgical, e.g. cosmetic applications) and may include robotic systems having guidance. In some examples instruments can be guided and controlled by the robotic system during a medical or cosmetic procedure. For example, the devices described herein may be used through one or more operating channels of a robotic system.

The apparatuses described herein may include elongate applicator tools (e.g., applicator catheters) that may be inserted into a body lumen, including but not limited to an esophagus, ear, nose, throat, trachea, pharynx, larynx, bronchi, uterus, fallopian tubes, stomach, small intestine, large intestine, duodenum, colon, blood vessels. These applicator tools may include an elongate body extending in a proximal-to-distal direction. One or more (e.g., a plurality) of electrodes configured for the delivery of high voltage, sub-microsecond electrical pulses (nanosecond pulsing, to deliver a nanosecond pulsed electric field to a target tissue) may be present at an end region of the elongate body. The elongate body may include one or more channels, including a channel enclosing electrical connectors/contacts for the electrodes and one or more lumen for a vacuum. In some cases, the body may include a channel or lumen for delivery of a material (e.g., a fluid material, including a therapeutic material) from the applicator tool. The applicator tool may be configured to include in some embodiments a removable or disposable tip, which may be removably coupled to a handle and through the handle to pulse generator configured to generate, for example, the nanosecond pulsed energy. Alternatively, in some cases the applicator tool (also referred herein as apparatus or device) may be configured to couple to the pulse generator directly, without the need for an additional handle.

The applicator tools (e.g., catheters) described herein may be flexible enough to easily bend within the confines of a tortuous body lumen but may have sufficient column strength so that it may be driven and inserted into the body lumen from the outer opening (e.g., esophagus, nose, throat, rectum, etc.), without collapsing or kinking. In some examples the walls of the apparatus may be reinforced with a support material (e.g., wire, braid, etc.).

When the applicator tool is coupled to a handle, the handle (proximal handle) may include one or more controls for activating one or more of: vacuum, electrodes, and/or an expander for reducing wrinkles in a wall of the lumen into which the applicator tool has been inserted.

According to one example, apparatuses described herein comprise medical devices and instruments for use in procedures inserting the applicator tools into an otolaryngological lumen. These apparatuses (e.g., the applicator tools) may be introduced through a natural opening (nostril, throat, ear canal) and can be used to treat, for example, mucosal epithelium, nasal polyps, mucous membrane papillomas, vocal fold carcinomas, and other head and neck diseases, lesions and tumors.

Any of these apparatuses may include a scope (e.g., camera, etc.) or may be configured for use with a scope. For example, the apparatus may include a fiber optic for illuminating and/or imaging the surrounding region within the lumen of the body into which the apparatus is inserted. This may allow a user (e.g., physician) to image before, during and/or after a procedure within a body lumen using the applicator tool, such as applying nanosecond pulsing to treat a target tissue (e.g., tumor, etc.). Examples of both applicator tools and methods of using them to treat indications are described in greater detail below. Thus, any of these apparatuses may, but does not necessarily have to, include one or more visualization components (e.g., a fiber optic, camera, lenses, filters, etc.). Thus, any of the apparatuses described herein may be configured as a scope. An elongate applicator tool, such as a catheter as described herein, including (but not limited to) those configured as a laparoscope, may include a tip region at or near the distal end of the applicator tool having a plurality of electrodes on one side and/or circumferential, for delivery of the electric treatment (e.g., delivery of a nanosecond pulsed electric field).

In some cases, the electrodes may be recessed into the outer surface of the applicator tool, flush with an outer surface of the applicator tool, or may stand proud of the applicator tool outer surface. Any of these applicator tools may include a suction or vacuum inlet at or adjacent to the location of the electrodes, to assist in making contact with the tissue by applying negative pressure to draw the tissue against the electrode(s).

In some examples the applicator tool may include one or more contact projections (e.g., contact plates, contact posts, balloons, etc.) that may be manipulated from the proximal end of the applicator tool and/or the handle to which the applicator tool is coupled. The contact projection may typically make contact with the wall of the lumen into which the applicator tool is inserted so that it may apply tension, e.g., to flatten the region of the wall near the electrodes to enhance access and contact with the tissue against the electrodes. For example, the contact projection may be an inflatable element (e.g., balloon) or a mechanical element (e.g., a pair of plates or arms that may be moved closer or further apart, near the electrodes. In some examples the contact projection(s) flank the electrodes and may help flatten or stretch out the region of the target tissue, holding it in tension. The contact projections may be retractable/removable into the catheter, or simply relative to the catheter.

The apparatuses described herein may generally be configured safely and reliably to deliver various energy modalities, in some implementations including RF energy, or pulsed electric fields, including microsecond, nanosecond, picosecond, etc. pulses. The methods and apparatuses described herein may be configured specifically to apply non-thermal energy, such as pulsed, sub-microsecond (e.g., nanosecond, between 1000 ns and 0.1 ns, etc.). Alternatively, any of these apparatuses may be adapted for use with other energy modalities, including thermal modalities (e.g., RF energy).

For example, in some implementations, electric field has a pulse width of between 0.1 nanoseconds (ns) and less than 1000 nanoseconds, or shorter, such as 1 picosecond, which may be referred to as sub-microsecond pulsed electric field. This pulsed energy may have high peak voltages, such as 1 to 5 kilovolts per centimeter (kV/cm), 10 kV/cm, 20 kV/cm, 100 kV/cm or higher. Treatment of biological cells may use a multitude of periodic pulses at a frequency ranging from 0.1 per second (Hz) to 10 MHz (e.g., between 0.1 Hz to 8 MHz, between 1 kHz to 5 MHz, between 10 kHz to 10 MHz, etc.), and may trigger apoptosis, for example, in the diseased tissue or abnormal growth, such as cancerous, precancerous or benign tumors and lesions. Selective treatment of such tumors with high voltage, sub-microsecond pulsed energy can induce apoptosis within the tumor cells without substantially affecting normal cells in the surrounding tissue due to its non-thermal nature. In some examples the method and/or apparatus may be configured to selectively treat some cells but not other. Using sub-microsecond pulsing as described herein, the selection may be achieved by adjusting the frequency and/or intensity of the applied pulsing.

A subject may be a patient (human or non-human, including animals). A user may operate the apparatuses described herein on a subject. The user may be a physician (doctor, surgeon, etc.), medical technician, nurse, or other care provider. An apparatus may refer to a system, device, or sub-system.

Thus, the application of high voltage, fast (e.g., microsecond or sub-microsecond) electrical pulses may include applying a train of electrical pulses having a pulse width, for example, of between 0.1 nanoseconds (ns) and 1000 nanoseconds. Applying high voltage, fast electrical pulses may include applying a train of sub-microsecond electrical pulses having peak voltages of between, for example, 1 kilovolts per centimeter (kV/cm) and 500 kV/cm. Applying high voltage, fast electrical pulses may include applying a train of sub-microsecond electrical pulses at a frequency, for example, of between 0.1 per second (Hz) to 10,000 Hz.

For example, described herein are apparatuses for treating tissue. Any appropriate tissue may be treated, including tissue of one or more walls of a lumen or portion of a lumen (e.g., including but not limited to pharynx, esophagus, stomach, small intestine, large intestine, etc.). In some examples the apparatuses and methods described herein may be used to treat one or more of these tissues, as part of a treatment therapy. In some examples the therapy may be for treatment of cancer. In some example the methods and apparatuses described herein may be used to treat a tumor or tumors.

Any of these apparatuses may be used with and/or may include a pulse generator. For example, described herein are systems for treating tissue that may include: an elongate applicator tool as described herein (e.g., an elongate body having a distal end region that articulates, from which one or more electrodes are configured to extend), a connector, e.g., a high voltage connector adapted to couple the elongate applicator tool to a pulse generator; and a pulse generator configured to generate a plurality of electrical pulses having amplitude of at least 0.1 kV and a duration of less than 1000 nanoseconds, the pulse generator may include a port configured to connect to a high voltage connector and thus to the electrodes.

Also described herein are methods of using any of the apparatuses (and systems that include such apparatuses) to treat tissue. Generally the elongate applicator tools described herein may be configured to treat tissue within a body by delivering, through the one or more electrodes, one or a train of high voltage, fast (e.g., sub-microsecond, nanosecond, picosecond) pulses. For example, described herein are methods of treating tissue, the method comprising: inserting a distal end of an elongate applicator tool into a lumen (e.g., a target anatomical structure, for example, otolaryngological structure, such as an ear, nose, or throat, including anatomical structures, such as turbinates, tonsils, tongue, soft palate, parotid glands, esophageal submucosal glands and other submucosal glandular structures; a gastrointestinal tract, e.g., stomach, small intestine, large intestine, duodenum, colon, etc., including, but not limited to the esophagus; a respiratory tract, including the trachea, pharynx, larynx, and bronchi, etc.), wherein the elongate applicator tool comprises at least two electrodes at a distal end region. The method may comprise viewing, through the elongate applicator tool, a wall of the lumen to identify a target region; and applying (e.g., using a proximal control, e.g., on handle) a plurality of electrical pulses having an amplitude of greater than 0.1 kV and a duration of less than 1000 nanoseconds from the at least two electrodes to the target region. In some examples, after identifying the target region, suction may be applied around or adjacent to the at least two electrodes at the distal end of the applicator tool. In some examples, an extendable contact projection, which may be inflatable or mechanically deployed, may be actuated from the proximal end of the applicator tool (e.g., the handle), to smooth the region of the tissue adjacent to the at least two electrodes.

In general, any of these apparatuses may be used in conjunction with or be a part of the system that includes a pulse generator configured to generate a plurality of electrical pulses having amplitude of at least 0.1 kV and a duration of less than 1000 nanoseconds, the pulse generator may be configured to connect to the apparatus with a high voltage connector.

Any of these apparatuses may include a suction inlet adjacent to the first and second electrodes.

As mentioned, any of these apparatuses may be configured so that the proximal end of the applicator tool is adapted to be coupled to a robotic or movable arm, for example, for computer-controlled activation of the first and second electrodes. Alternatively or additionally the proximal end of the applicator tool may be adapted to couple to a handle of the pulse generator which may in turn by adapted for connection to a robotic arm.

For example, any of the apparatuses described herein may include a distal tip region having a plurality of circumferentially arranged electrodes. For example, an elongate applicator apparatus may include: an elongate shaft; a distal tip region at a distal end of the elongate shaft, wherein the distal tip region comprises: a plurality of electrodes arranged circumferentially around the distal tip region; and one or more openings into a vacuum port adjacent to or underneath the plurality of electrodes; and, optionally, a high voltage connector at a proximal end of the elongate shaft configured to couple to a pulse generator.

In any of these apparatuses and methods, the plurality of electrodes may extend longitudinally and may be spaced circumferentially. Alternatively, the plurality of electrodes may extend circumferentially and may be spaced longitudinally. Further, in any of these apparatuses the electrodes of the plurality of electrodes may be separated by a conductive spacer. For example, a conductivity of the conductive spacer may be greater than or equal to a conductivity of a tissue of a treatment area (e.g., up to 10 times the conductivity of the tissue, up to 100 times the conductivity of the tissue).

Any of these apparatuses may include a pulse generator configured to generate a plurality of electrical pulses having amplitude of at least 0.1 kV and a duration of less than 1000 nanoseconds, the pulse generator may include a port configured to connect to the electrodes, e.g., via a high voltage connector. The pulse generator may include a handle; the high voltage connector may be configured to mate with the connector.

The plurality of electrodes may comprise one or more of: spring electrodes, plate electrodes, bar electrodes or wire electrodes. In some examples electrodes of the plurality of electrodes are formed from a flex circuit material. In some examples the plurality of electrodes may be configured to be energized simultaneously or multiplexed together. The spring electrodes may be a coil-shaped electrode that may expand and contract; this configuration may be particularly useful for circumferential electrodes in which the spring electrode extends around the circumference of the distal end region and can expand and contract. The spring electrode may also be useful when applying suction through the electrode, e.g., to increase contact with the tissue.

In any of these apparatuses, the distal tip region may have a diameter of between about 2 mm to about 30 mm. These apparatuses may be configured for esophageal use and may be dimensioned accordingly. For example, the elongate body may be between 1 cm and 50 cm (or more) long, depending on the relevant treatment area. Thus, the elongate applicator tool may be an esophageal applicator tool configured for insertion into an esophagus.

The apparatuses and methods described herein may be specifically configured for treating Barrett's Esophagus.

In any of these apparatuses, the distal tip region may comprise a cylindrical or cone-shaped body having an open channel configured to receive a medical scope (e.g., an endoscope, bronchoscope, etc.) therethrough. In some cases just the distal tip region may engage with the medical scope, similarly to a rapid-exchange device. For example, the elongate shaft may have a diameter that is less than the diameter of the distal tip region and it may be configured to extend co-axial or adjacent to the medical scope when the medical scope is received in the open channel. In any of these apparatuses the channel may extend just over a short distal region (e.g., the distal tip region) of the apparatus. This may allow other devices to be inserted into the channel while in the body.

In any of these apparatuses the distal tip region may be configured to expand to increase the outer diameter of the distal tip region. For example, the distal tip region may comprise an expandable member, such as a balloon. In some examples the distal trip region may include an expandable member, such as a frame (e.g., a mechanical frame). The plurality of electrodes may be configured to expand as the distal tip region expands. For example, the plurality of electrodes may comprise one or more of: spring electrodes, overlap band electrodes, or wire electrodes (e.g., sinusoidal or mesh wire electrodes) just to name a few, each of which may expand or contract with the outer diameter of the distal tip region.

Thus, any of these apparatuses may include a distal end that is adapted to engage with a medical scope. In some examples a medical scope may pass through a lumen extending the length of the elongate member and the distal tip region; in some examples (as mentioned above) the medical scope may engage with just the distal tip region. For example, an elongate applicator apparatus may include: an elongate shaft; a distal tip region at a distal end of the elongate shaft, wherein the distal tip region comprises a channel configured to engage a medical scope, the distal tip region further comprising: a plurality of electrodes arranged along the distal tip region; and, optionally, a high voltage connector at a proximal end of the elongate shaft configured to couple to a pulse generator. In some examples, the apparatus may include one or more openings into a vacuum port adjacent to or underneath the plurality of electrodes. These apparatuses may include any of the features described in reference to various examples of the present disclosure.

For example, the distal tip region may include a cylindrical body having an open channel configured to receive a medical scope therethrough. In any of these apparatuses, the channel may be configured as a clip that may couple with (e.g., snap onto) the medical scope. In general, the medical scopes described herein may be elongate medical scopes for imaging an internal region of the body, including internal lumen. The medical scope may be flexible, and may include imaging, lighting and one or more channels for passing material (e.g., fluid, instruments, etc.). Examples of medical scopes may include, but are not limited to endoscopes, bronchoscopes, gastroscopes, colonoscopes, laryngoscopes, cystoscopes, enteroscopes, duodenoscopes, uteroscopes, hysteroscopes, etc. These scopes may all be referred to herein as medical scopes or, for convenience, simply “endoscopes.”

Also described herein are apparatuses that include one or more electrodes that may be expandable. In some examples the electrode may expand (and/or contract) as the distal end of the apparatus expands and contracts. Alternatively or additionally, the one or more electrodes may expand and contract relative to the distal end of the apparatus. For example, an elongate applicator apparatus may include: an elongate shaft; a distal tip region at a distal end of the elongate shaft, wherein the distal tip region comprises a channel configured to engage a medical scope, at least a portion of the distal tip region being expandable or comprising an expandable member; a plurality of radially expandable electrodes arranged along an expandable portion of the distal tip region; and, optionally, a high voltage connector at a proximal end of the elongate shaft configured to couple to a pulse generator.

Any of these apparatuses may include one or more expandable electrodes, for example, on the outer surface of the distal tip region that expand separately (and in some cases eccentrically) relative to the distal tip region. For example, an elongate applicator apparatus may include: an elongate shaft; an actuating shaft extending within or adjacent to the elongate shaft; a distal tip region at a distal end of the elongate shaft comprising: one or more overlap band electrodes, each comprising a strip of conductive material wrapped over itself and configured to radially expand to a diameter that is greater than the outer diameter of the distal tip region as an actuating shaft is rotated. The strip of conductive material may form or may be attached to an electrodes support. In some examples, the applicator apparatus may comprise a high voltage connector at a proximal end of the elongate shaft configured to couple to a pulse generator.

For example, a first portion of the one or more overlap band electrodes may be coupled to the actuating shaft while a second portion of the one or more overlap band electrodes may be coupled to the distal tip region so that rotation of the inner shaft drives expansion or compression of the one or more overlap band electrodes. The expansion or compression may be eccentric. The distal tip region may include a channel configured to engage a medical scope (e.g., an endoscope). In some examples, multiple bands of overlap band electrodes or electrode support structures may be spaced apart along the length of the distal tip region of the applicator apparatus. While each band may be expanded as described above, some or all of the bands may have different compliance, so that when expanded they may each differently conform to the portions of the wall of the relevant anatomical structure providing better conformity, especially when such structure has irregular shape or changing diameter/circumference.

Also described herein are methods of operation or methods of using an applicator apparatus including an overlap band electrode. For example, a method may include: inserting a distal end of an elongate applicator into a body lumen, wherein the elongate applicator comprises one or more overlap band electrodes; rotating an actuating shaft of the elongate applicator to radially expand the one or more overlap band electrodes relative to a distal tip region at a distal end of the elongate applicator; and applying an electrical treatment to a target region on or through the body lumen.

Any of these methods may include rotating the actuating shaft by rotating one end of the one or more overlap band electrodes relative to the distal tip region. For example, rotating may include rotating one end of an electrode support onto which the one or more overlap band electrodes is coupled. In some examples, the other end of the one or more overlap band electrodes and/or the electrode support may be free to slide over itself. In some examples, rotating the actuating shaft comprises rotating the actuating shaft relative to an elongate body of the elongate applicator.

Any of these methods may include positioning the distal tip region within the body lumen adjacent to a target region (e.g., on a wall) of the body lumen. As mentioned above, the body lumen may be an esophagus. In some examples the target region is a mucosal epithelium of an ear, nose, mouth, tongue, turbinate, tonsils, soft palate, submucosal glands, or parotid glands. The target region may be a mucosal epithelium of a stomach, small intestine, large intestine, duodenum, colon, fallopian tubes, etc. The target region may be a mucosal epithelium of a trachea, pharynx, larynx, or bronchi. In some examples the target region may also be the wall of a structure, for example, an intima of the blood vessels, the endometrium of the uterus and other structures.

For example, described herein are methods of treating Barrett's esophagus using an apparatus as described herein. For example, a method of treating Barrett's esophagus may include: inserting a distal end of an applicator tool into a Barrett's esophagus of a subject; expanding an electrode assembly comprising one or more electrodes at the distal end of the applicator tool to contact a full circumference of a region of the esophagus; and treating Barrett's esophagus by applying a plurality of electrical pulses having an amplitude of greater than 0.1 kV and a duration of less than 1000 nanoseconds to the full circumference of the region of esophagus.

Expanding the electrode assembly may include rotating an actuating shaft of the elongate applicator to radially expand one or more radial electrodes. In some examples, expanding the electrode assembly comprises expanding one or more radially continuous electrodes. For example, expanding the electrode assembly may include expanding one or more overlap band electrodes relative to a distal tip region at the distal end of the elongate applicator. In some examples, expanding the electrode assembly comprises expanding a basket at the distal end of the applicator comprising a plurality of electrodes arranged circumferentially around the basket.

Any of these methods may include applying suction from beneath and/or beside the one or more electrodes on the distal end region of the applicator tool to secure the circumferential region of the esophagus to the one or more electrodes.

In any of the methods and apparatuses described herein, the tissue to be treated, being treated or already treated may be directly or indirectly visualized. A scope may be used with (and/or integrated into or passed through) the applicator tool. For example, any of these methods may include directly visualizing the esophagus using a scope applied through the applicator tool.

In some examples, the method may include radially constricting the electrode assembly and moving the distal end of the applicator longitudinally within the esophagus to treat a second circumferential region. Such treatment of the full circumference of the second (and additional) longitudinal areas of esophagus may be repeated, needed, for example, when the area affected by Barrett's epithelium is longer than a treatment size of the electrode assembly. However, the entire area of the Barrett's esophagus may be treated with the same applicator tool.

All of the methods and apparatuses described herein, in any combination, are herein contemplated and can be used to achieve the benefits as described herein.

### BRIEF DESCRIPTION OF THE DRAWINGS

The novel features of the present disclosure are set forth with particularity in the claims that follow. A better understanding of the features and advantages of the present disclosure will be obtained by reference to the following detailed description that sets forth illustrative examples, in which the principles of the disclosure are utilized, and the accompanying drawings of which:

FIG. 1 illustrates one example of a system, including an elongate applicator tool of the present disclosure and a pulse generator, for delivery of high voltage, fast pulsed electrical energy.

FIG. 2 shows one example of an applicator tool configured for use in a body lumen (e.g., esophagus) as described herein.

FIGS. 3A-3B show front and side views, respectively, of another example of an applicator tool as described herein.

FIGS. 4A-4C show further examples of applicators of the present disclosure. FIG. 4B shows an enlarged view of the tip of FIG. 4A, while FIG. 4C is an even further enlarged view of the electrode and suction/vacuum port region of FIG. 4A-4B.

FIG. 5 shows examples of the treatment tips of the applicator tools configured to different dimensions, for use as an esophageal apparatus.

FIG. 6 schematically illustrates one example of a section through a tip of a tool, showing sections through spring electrodes and vacuum regions.

FIG. 7 is an example of a distal tip region of an applicator tool including circumferential electrode configuration.

FIGS. 8A-8B illustrate another example of an apparatus including a distal tip region having a plurality of circumferentially-arranged electrodes. FIG. 8A shows the apparatus including the distal end and the proximal connector region; FIG. 8B shows an enlarged view of the distal tip region of the apparatus shown in FIG. 8A.

FIG. 9A is a drawing of another apparatus configured for delivery of pulsed electrical energy within a body lumen (such as but not limited to the esophagus). The example shown in FIG. 9A is with spring electrodes similar to that shown in FIGS. 8A-8B.

FIG. 9B shows an alternative example of an apparatus similar to that shown in FIG. 9A but with plate electrodes.

FIGS. 10A-10C illustrate another example of an elongate applicator apparatus. The apparatus shown in FIGS. 10A-10C is particularly well adapted for use with an endoscope and is configured to clip on to the end region of an endoscope. FIG. 10A shows an example of the apparatus unclamped from the endoscope, while FIG. 10B shows the channel region of the apparatus that may engage with the outer diameter of the endoscope. FIG. 10C shows the apparatus clipped onto an endoscope.

FIGS. 11A-11C illustrates another example of an apparatus with a clip-on distal tip region configured to engage with a distal end region of an endoscope. FIG. 11A shows the apparatus by itself (not coupled to an endoscope) while FIGS. 11B and 11C illustrate the apparatus of FIG. 11A snapped onto an endoscope.

FIGS. 12A-12C show another example of an apparatus comprising a plurality of circumferential electrodes that are formed, by example, of a material similar to a flexible printed circuit having a distal tip region that engages with the endoscope, as illustrated in FIGS. 12A-12B. FIG. 12C shows an enlarged view of the distal tip region.

FIGS. 13A-13B illustrate another example of an apparatus having a distal tip region configured to engage with an endoscope and having expandable electrodes. As shown in FIG. 13A-13B circumferential electrodes on the distal tip region expand radially (e.g., to better contact the lumen walls before/during treatment).

FIGS. 14A-14E illustrate operation of an apparatus similar to that shown in FIGS. 13A-13B. In FIG. 14A the apparatus is shown with the distal tip region un-expanded and without an endoscope. FIG. 14B shows the apparatus engaging with a distal end region of an endoscope.

FIGS. 14C and 14D show, respectively, a prospective and a side view of the apparatus with the distal tip region and electrode radially expanded. FIG. 14E illustrates the apparatus with the distal tip region and electrode radial expanded and engaging with an endoscope.

FIGS. 15A-15D illustrate the operation of one example of an overlap band electrode that may expand and contract either with the distal tip region or independently of the distal tip region. FIGS. 15A-15B show the overlap band electrode in the constricted configuration, having a smaller diameter. FIGS. 15C-15D illustrate the overlap band electrode in an expanded configuration, having a larger diameter.

FIGS. 16A-16F illustrate another example of an apparatus having a radially expandable distal tip region that is also configured to engage with an endoscope, as shown in FIGS. 16B, 16C and 16E. The apparatus shown in FIGS. 16A-16F includes a plurality of expandable spring (or helical coil) electrodes.

FIGS. 17A-17B illustrate another example of an expandable (e.g., mesh) electrode that may be used with any of the apparatuses described herein.

FIG. 18 is an example of an apparatus including an expandable distal tip region (including a balloon/inflatable distal tip region).

FIGS. 19A-19B show an example of an apparatus as described herein including a radial expandable overlap band electrode that may expand independently of the distal tip region to which it is attached. FIG. 19A shows the overlap band electrode in the un-expanded configuration while FIG. 19B shows the overlap band electrode in an expanded configuration.

FIG. 20 shows an example of an apparatus similar to that shown in FIGS. 19A-19B configured to engage an endoscope.

FIG. 21 is a diagram showing various body lumens/structures that may be treated with the methods and apparatuses (e.g., treatment lumen) described herein.

FIGS. 22A-22D illustrate nanosecond pulsed field treatment of a region of porcine esophagus. FIG. 22A shows an untreated control. FIG. 22B shows a region 12 hours after treating with 2.3 J of nanosecond pulsed electric field. FIG. 22C shows a region 12 hours after treating with 5.1 J of nanosecond pulsed electric field. FIG. 22D shows a region 12 hours after treating with 10.9 J of nanosecond pulsed electric field. These tissue sections were stained with hematoxylin and eosin and captured at 4× magnification.

FIGS. 23A-23D are similar to FIGS. 22A-22D but have been stained with Movat's pentachrome and captured at 10× magnification. In FIG. 23A the control (untreated) esophageal section is shown. FIGS. 23B, 23C and 23D show 2.3 J, 5.1 J and 10.9 J, respectively, of nanosecond pulsed electric fields.

FIG. 24A schematically illustrates one example of a method of treating a lumen (target tissue) as described herein.

FIG. 24B schematically illustrates another example of a method of treating a lumen as described herein.

FIG. 25 schematically illustrates an example of a method of treating Barrett's esophagus as described herein.

### DETAILED DESCRIPTION

Described herein are elongate applicator tools adapted to be inserted into a body lumen or structure, and in particular, into a lumen of an otolaryngological structure (such as an ear, nose, or throat, including anatomical structures, such as turbinates, tonsils, tongue, soft palate, parotid glands), a gastrointestinal tract (e.g., stomach, small intestine, large intestine, duodenum, colon, etc., including the esophagus), and/or a respiratory tract (e.g., trachea, pharynx, larynx, and bronchi), to deliver high voltage (e.g., microsecond, nanosecond, picosecond, etc.) electrical energy to target tissue without damaging surrounding (non-target) tissue. Some of the examples of the apparatuses of the present disclosure may be implemented using RF energy.

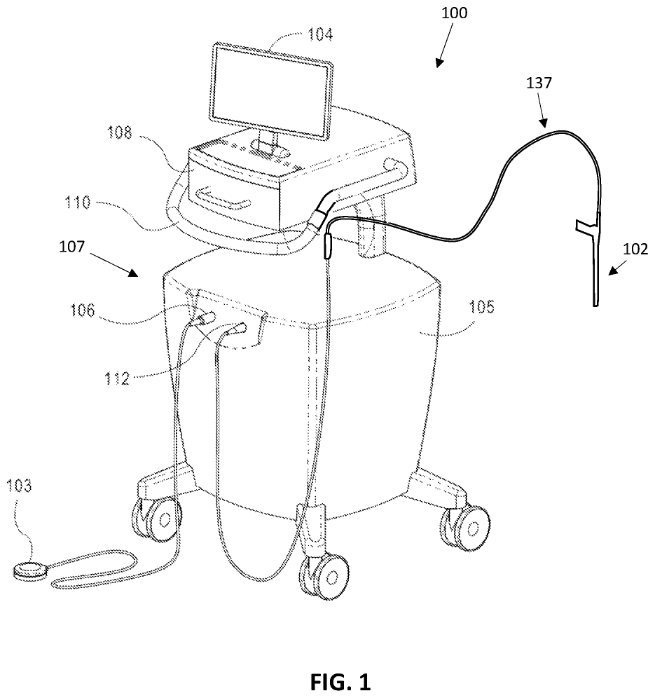

FIG. 1 illustrates one example of a system 100 (also referred to herein as a high voltage system or a sub-microsecond generation system) for delivering high voltage, fast pulses of electrical energy that may include an elongate applicator tool 102, a pulse generator 107, footswitch 103, and user interface 104. Footswitch 103 is connected to housing 105 (which may enclose the electronic components) through a cable and connector 106. The elongate applicator tool 102 may include electrodes and is connected to housing 105 and the electronic components therein through a cable 137 and high voltage connector 112. The high voltage system 100 may also include a handle 110 and storage drawer 108. The system 100 may also include a holder (e.g., holster, carrier, etc.) (not shown) which may be configured to hold the elongate applicator tool 102.

In some cases, the applicator tool includes imaging, such as one or more cameras and/or fiber optics at or near the distal end of the applicator tool. The camera(s) may be forward-facing and/or side facing. The apparatus 100 may be configured to display images (in real time, and/or recorded) taken by the applicator tool, in order to identify the target region(s).

A human operator may select a number of pulses, amplitude, pulse duration, and frequency information, for example by inputting such parameters into a numeric keypad or a touch screen of interface 104. In some examples, the pulse width can be varied. A microcontroller may send signals to pulse control elements within system 100. In some examples, fiber optic cables are used which allow control signaling while also electrically isolating the contents of the metal cabinet with sub-microsecond pulse generation system 100, e.g., the high voltage circuit, from the outside. In order to further electrically isolate the system, system 100 may be battery powered instead of being powered from a wall outlet.

The elongate applicator tool may be hand-held (e.g., by a user) or it can be affixed to a movable arm of a robotic system, and its operation may be at least partially automated or fully automated, including computer controlled.

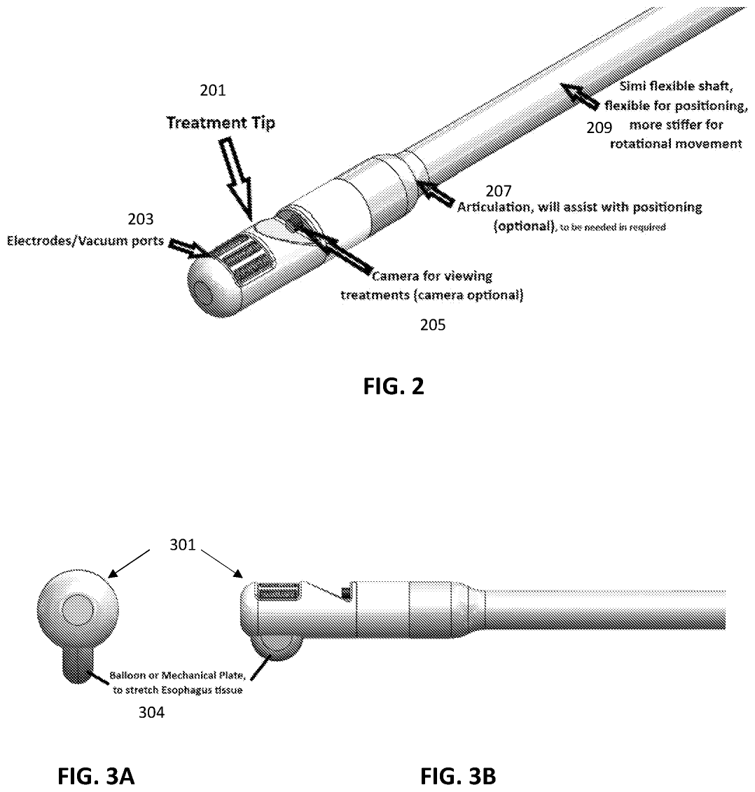

FIG. 2 illustrates one example of a distal tip region of an applicator tool as described herein. In this example, the distal end region of the applicator tool includes a plurality of electrodes (shown just as an example as longitudinally arranged coil electrodes 203) on the distal treatment tip region 201. The applicator tool may also include a camera 205 for viewing treatments; the camera (while optional) is formed on the tip region to look at the region of the lumen or anatomical structure that is adjacent to the electrodes. An illumination source (e.g., fiber optic, LED, etc.) may also be included. The applicator tool of at least a portion of the applicator tool may also rotate, pivot or articulate, e.g., around the long axis 207. This rotation/pivoting/articulation may be controlled proximally by an input. The applicator tool also includes a shaft 209, shown here as a semi-flexible shaft that may be sufficiently stiff to prevent twisting (e.g., rotation), while allowing bending. The distal treatment tip region 201 may be slightly larger than the elongate shaft 209. In this example one or more vacuum ports underlie the electrodes on the tip, so that suction may be applied to draw the tissue against the electrodes.

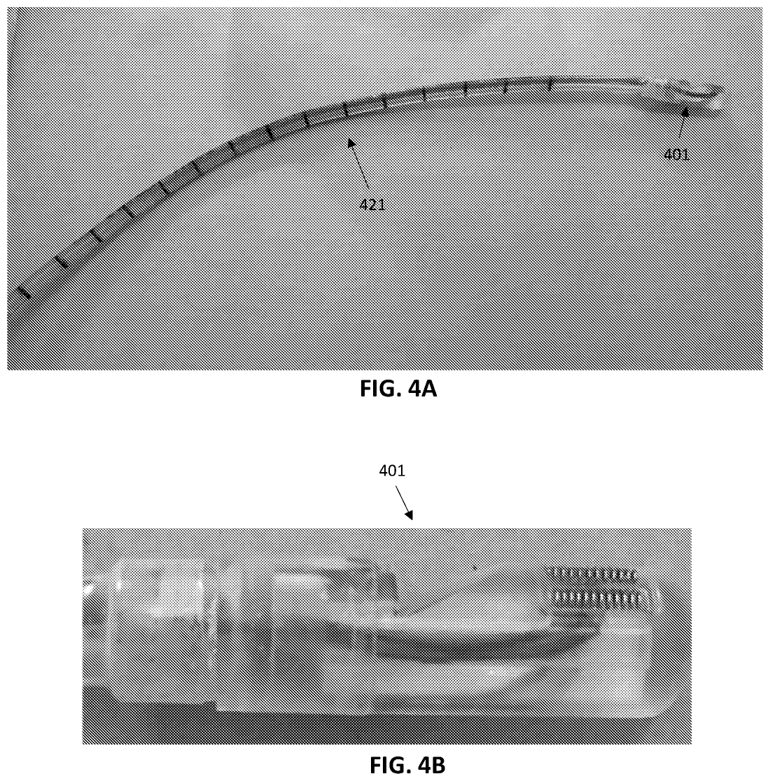

FIGS. 3A-3B illustrate another example of an applicator tool similar to that shown in FIG. 2, but with a contact projection 304 on the distal treatment tip region 301. In this example, the contact projections are shown as a pair of mechanical arms or projections that may be expanded outward, and/or rotated around the circumference of the tip, prior to applying the vacuum and performing the treatments. Alternatively, or additionally, a contact projection (which may be also referred to as tissue-expanding projection) such as a balloon or other mechanical structure (e.g., projection) may expand outward to smooth, tighten, or otherwise mechanically prepare the target tissue (for example, esophagus tissue) prior to applying the vacuum and performing the treatments. Any tissue-expanding projection may be used, including but not limited to one or more balloon(s), arm(s), frame(s), or other type of mechanical structure or mechanism that may project from the device to mechanically prepare the tissue, e.g., by holding it taut, in preparation for contact by the energy applying element(s), such as electrodes, of the device. These structures may be generally referred to as tissue-expanding projections or contact projections. By expanding the tissue at or near the electrodes the projections may remove any natural wrinkles in the tissue, which may otherwise cause incomplete treatment. It should be understood that esophagus tissue is provided as an example only, and the contact/tissue expansion projections may be used with various anatomical structures mentioned in the present disclosure, as applicable, to smooth the target tissue. In some examples the tissue-expanding projection, such as contact projections, may extend on either side of the electrodes, framing the region of the tissue to be treated on at least two sides, with the tissue held taught between then. In other examples, the tissue-expanding projections may be on the opposite side of the tip from the electrodes.

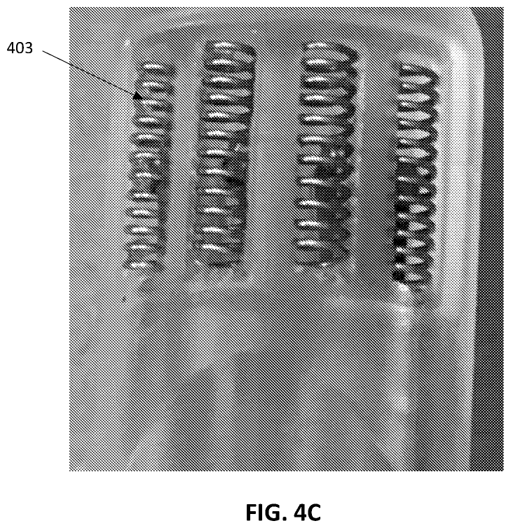

FIG. 4A is an example of an applicator tool (e.g., an endoscopic device or a catheter) 421, configured for use in an esophagus. The applicator tool may be sized and dimensioned based on the expected (e.g., average) human esophagus dimensions. An enlarged view of the tip region 401 is shown in FIG. 4B. As shown in FIG. 4B, the tip region includes a plurality of coil electrodes that are adjacent to a vacuum port. In FIG. 4B, four coil (e.g., spring) electrodes are shown. This esophagus applicator tool could be configured with multiple different electrode configurations such as 2, 3, 4 or more electrodes. Although in FIGS. 2-4B the electrodes shown are coil (also referred to as spring) electrodes, other electrode types may be used, including but not limited to various surface electrodes, such as plates, bars, or wire electrodes. In some applications needles electrodes may be used as well. As mentioned above, suction (e.g., via a vacuum) may be pulled through and around the electrodes to tightly hold the tissue in contact with the electrodes. FIG. 4C shows a slightly enlarged view of the spring electrodes 403 of this example. Although these figures show surface electrodes configured as coil/spring electrodes any surface electrode may be used.

The use of suction is optional. In some cases, suction is not applied. In some examples one or more tissue-expanding projection may be used instead or in addition to suction. Suction may be applied to secure contact between the tissue and the electrode in some cases.

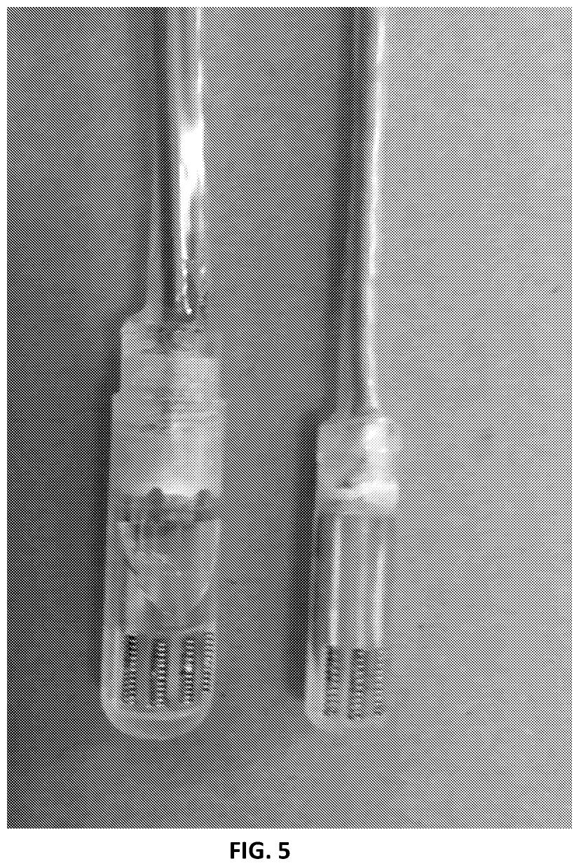

FIG. 5 shows another example of an applicator tool as described herein. In FIG. 5, two sizes of treatment tips are shown. In general, the treatment tips described herein could be designed in multiple different diameters. In FIG. 5 the tip on the left is a 16 mm (diameter) tip, while the tip on the right is a 12 mm (diameter) tip.

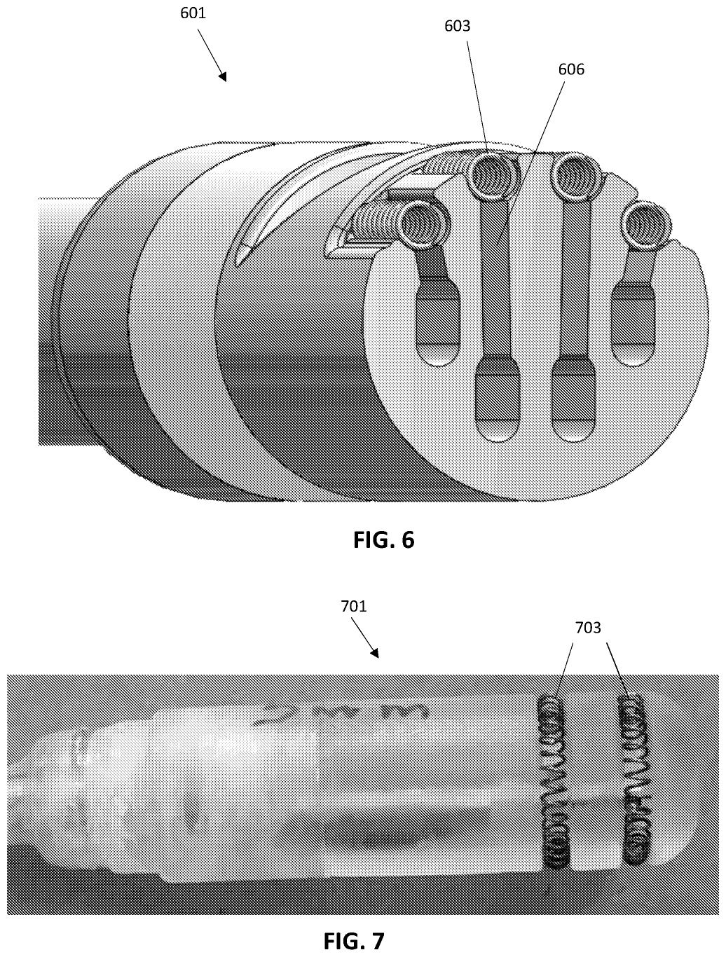

A schematic of another example of a tip of an applicator tool is shown in FIG. 6. In this example, a section through the tip 601 shows by example the spring electrodes 603 overlie a channel opening for suction (vacuum) 606. This tip example has a 16 mm tip diameter and shows the spring-type electrodes with the vacuum chambers below the springs.

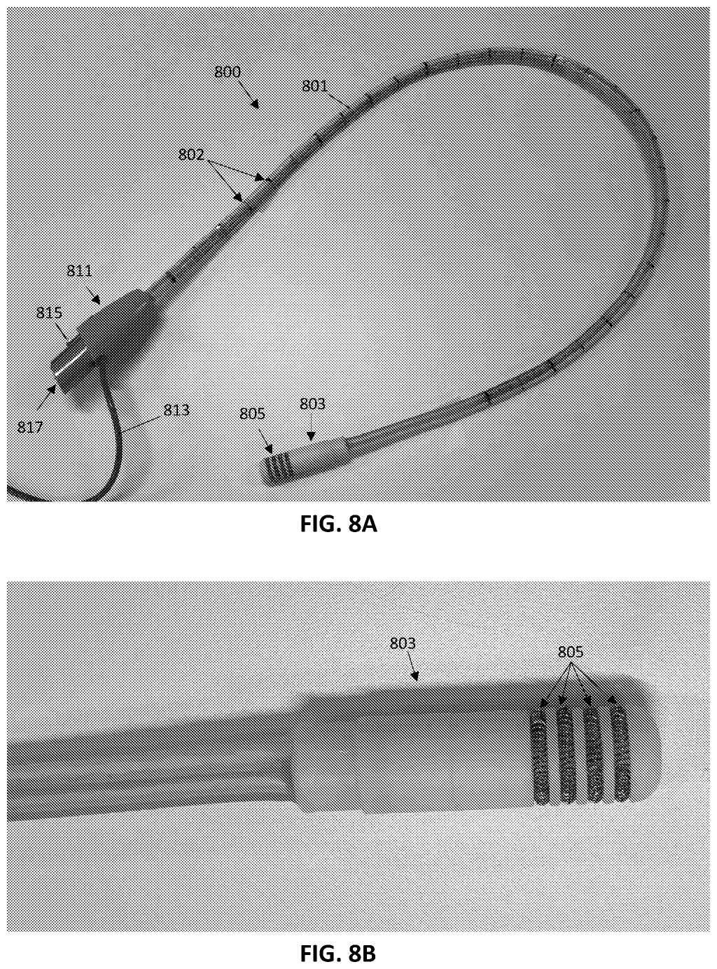

Another example of a tip end region 701 of an applicator tool is shown in FIG. 7. In this example, a circumferential spring electrode 703 is shown. The electrodes and/or tip can be configured differently (e.g., to have different shape configurations) such as the circular electrode configuration shown in FIG. 7, which may allow a full circumference of the lumen (e.g., esophagus lumen) to be treated.

The apparatus shown in FIG. 7 may be particularly helpful for treating a mucosal layer of a patient's duodenum, as described in greater detail below, in the context of a duodenal mucosal resurfacing procedure. This apparatus may be configured to perform full circumferential ablation or partial ablation.

### Otolaryngology Methods

In general, the methods and apparatuses described herein may use nanosecond field pulsing to treat, for example, benign and malignant lesions occurring on the epithelial surface or inner wall lining of various anatomical structures to trigger regulated cell death in targeted cells and allow for the treatment of any neoplastic growth inside and outside the body. For example, the methods and apparatuses described herein may be configured for the selective ablation and resurfacing of the mucosal epithelium of the ear, nose, or throat, including in anatomical structures, such as turbinates, tonsils, tongue, soft palate, parotid glands, esophagus, submucosal glandular structures, etc. using nanosecond pulsing. Nanosecond pulsing can be performed on benign or malignant tumors, such as nasal polyps, papillomas, vocal fold carcinoma, squamous cell carcinoma, and other head and neck cancers. For example, the methods and apparatuses described herein may be useful for nanosecond pulsing treatments focused on ablation of the mucosal epithelium and submucosal glands.

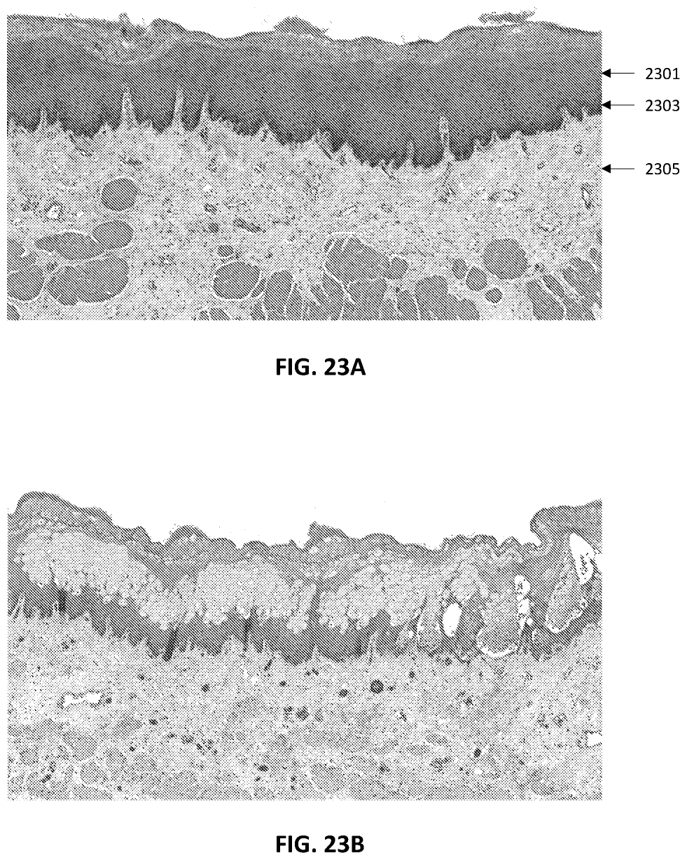

FIG. 21 is a diagram of a portion of a human body, showing by example multiple regions in which these apparatuses and method may be used, such as the pharynx, trachea, upper esophageal sphincter, esophagus, lower esophageal sphincter, diaphragm, stomach, etc. Nanosecond pulsing as described herein may be used for the ablation of the mucosal epithelium that may eliminate or reduce scar formation. For example, these methods and apparatuses may provide resurfacing of the mucosal epithelium or removal of tumor tissue from the ear, nose, or throat, including anatomical structures, such as turbinates, tonsils, tongue, soft palate, parotid glands, submucosal glands using nanosecond pulses. In addition (not shown in FIG. 21), apparatuses and methods of the present disclosure may be used for treatment of epithelium of reproductive structures, such as fallopian tubes, as well as the endometrium of the uterus, or intima of various blood vessels. Nanosecond pulsing treatment can be performed on benign or malignant tumors, such as nasal polyps, papillomas, vocal fold carcinoma, squamous cell carcinoma, and other head and neck cancers. Treatment of tumors on sensitive structures, such as laryngeal tumors is of particular interest. Commonly, these tumors are surgically excised by cordectomy or laryngectomy, which can cause a significant decline in a patient's quality of life. Surgical intervention is often balanced with concerns of a patient's ability to eat, breath, and speak post-treatment. Nanosecond pulsing using the applicator tools described herein may provide a distinct advantage in the cell-specific nature of this therapy, where abnormal growths of cellular mass can be eliminated by triggering regulated cell death mechanisms without impacting underlying structures. This may prevent collateral impact from treating tumors arising on cartilage rich structures.

For example, squamous papillomas are benign tumors that affect skin, mucosa of the oral cavity, aerodigestive tract, and auditory canal. As described above, nanosecond pulsing is cell-selective and therefore can be used to eliminate epithelial growths, such as papillomas. A distinct advantage of nanosecond pulsing is the induction of regulated cell death. This allows cellular structures to be treated and eliminated, without triggering a significant immunologic response that leads to the formation of a scar. In this example, a squamous papilloma would be treated with nanosecond pulsing, e.g., using any of the applicator tools described herein. As nanosecond pulsing does not affect acellular structures and causes regulated cell death, tumors could be removed without (or with very little) scar formation. This may reduce or eliminate the negative outcomes associated with aggressively treating these neoplastic tissues.

Vestibular schwannomas or acoustic neuromas are tumors that arise on the vestibular or cochlear nerves that run between the brain and ear. Pressure that results from the growth to these tumors can lead to hearing loss and issues with balance. These tumors can be treated by surgical intervention, which can result in permanent impairment of nerve function. Preliminary data suggests that nanosecond pulsing using an applicator tool as described herein may not affect nerve when treatment is performed along the axon. Therefore, nanosecond pulsing may be used for treatment of vestibular schwannomas or acoustic neuroma. In this capacity nanosecond pulsing could effectively eliminate the entire tumor burden, leaving the neural functions unharmed. Alternatively, nanosecond pulsing could be used as a means for margin control in conjunction with another therapy, such as surgical resection or radiation.

Cholesteatomas are skin-lined cysts that begin at the margin of the eardrum and invade the middle ear and mastoid. The growth of these cysts can be aggressive in nature and leads to earache, drainage, pressure, hearing loss, dizziness, and facial muscle weakness. This condition is treated by surgical intervention, where the growths are excised. Similar to the previous examples, cholesteatomas could be a target for nanosecond pulsing treatment using any of the applicator tools described herein. This would allow the physician to induce regulated cell death of the aggressive cells composing the disease, while leaving collateral structures intact. This treatment has potential to significantly reduce the overall negative impacts associated with surgery, while restoring a patient's quality of life.

For example, the methods and apparatuses described herein may be used for selectively inducing cell death in laryngeal papilloma cells without stimulating fibrosis or scar creation in the superficial lamina propria. In some methods a selective effect in the target tissue may be created while sparing the deeper layers with the combination of creating a stronger electric field in the target cells then in the deeper layers and optimizing nanosecond pulsing parameters so that deeper tissues are more robust against the electric treatment than the target tissue. This same method may be applied to treat other targets, e.g. SCC, carcinoma, etc. The same method may be used for other anatomical structures as well, e.g. tonsils, tongue, soft palate, trachea, esophagus, parotid and other gland, etc.

These methods and apparatuses may also be used for resurfacing of the turbinates. Turbinate tissue can become inflamed and hyperactive in people with severe allergies. Resurfacing them may reset their reactivity back to normal, and/or alleviate some of the respiratory difficulty.

The methods and apparatuses described herein may also be used for treatment of neurotologic lesions, such as acoustic neuroma and cholesteatoma, by inducing rapid cell death (e.g., selective apoptosis) in the target lesion while minimizing collateral damage to surrounding nerves, vessels, bones and other structures.

In some examples, an esophagus may be treated using nanosecond pulsing with vacuum assist. An applicator tool configured as an esophagus treatment device (e.g., configured as a catheter) can be used to perform nanosecond pulsing treatments on lesions and/or therapies throughout the inside of an esophagus. Some treatment applications include the treatment of throat lesions, GERD's, Barrett's esophagus, nodules, as well as other benign or cancerous abnormal skin growths that may affect the esophagus or tongue. For example, a physician may use an applicator tool for treating an esophagus by inserting the treatment tip portion of the nanosecond pulsing into the mouth, down the throat down into the patient's esophagus until the electrodes are in-line with the lesion or intended location. This may be confirmed using visualization; in some examples the applicator tool may include an imaging sensor on or near the distal end of the applicator tool, including near the electrodes. Thus, for positioning, the applicator tool may or may not have a working channel for a fiber optic scope. For example, see FIG. 2, above (showing a possible location within this device). Alternatively or additionally, the applicator tool used may be positioned using fluoroscopy. Once in position, the one or more contact projections (e.g., balloons or mechanical projections, such as plates, as described in FIGS. 2-3, above), may be expanded as shown in FIG. 3, which may stretch out any wrinkles in the esophagus tissue. Once the esophagus is stretched and the position is confirmed, a vacuum (suction) may be turned on, pulling the tissue tight against the electrode/vacuum ports, at which point the treatment can be performed. The applicator tool may also include a pressure relief port to allow the vacuum to be vented following the treatment to allow removal or repositioning of the treatment tips portion.

Similar methods may also be used (e.g., following similar steps) in the stomach and/the colon. The apparatus may be inserted rectally, for example.

Any appropriate nanosecond scale pulsing may be applied. For example, the pulse profiles may also include a rise and/or fall times for pulses that may be less than 20 ns, about 20 ns, about 25 ns, about 30 ns, about 40 ns, about 50 ns, about 60 ns, about 75 ns, or greater than 75 ns. In some examples, the pulse voltage may be less than 5 kV, about 5 kV, about 10 kV, about 15 kV, about 20 kV, about 25 kV, about 30 kV, or greater than 30 kV. In some examples, the current may be less than 10 A, about 10 A, about 25 A, about 40 A, about 50 A, about 60 A, about 75 A, about 100 A, about 125 A, about 150 A, about 175 A, about 200 A, or more than 200 A. In some examples, the pulse duration may be less than 10 ns, about 10 ns, about 15 ns, about 20 ns, about 25 ns, about 30 ns, about 40 ns, about 50 ns, about 60 ns, about 75 ns, about 100 ns, about 125 ns, about 150 ns, about 175 ns, about 200 ns, about 300 ns, about 400 ns, about 500 ns, about 750 ns, about 1 μs, about 2 μs, about 3 μs, about 4 μs, about 5 μs, or greater than 5 μs. The apparatuses (e.g., systems) described herein may include, in addition to the instrument (e.g., the elongate applicator tool), a pulse generator such as the one shown schematically in FIG. 1, configured to emit pulses in the sub-microsecond range.

In general, the systems of the present disclosure may comprise additional elements, such as power supplies, and/or a high voltage connector for safely connecting the elongate applicator tool device to a high voltage power source. As described above, these systems and devices are configured to apply high voltage, sub-microsecond pulsed electrical energy.

The high voltage pulsing elongate applicator tool may be any appropriate length (e.g., between about 4 inches and about 200 inches, between about 4 inches and about 100 inches, between about 4 inches and 30 inches long, between about 6 inches and 50 inches long, between about 6 inches and 25 inches long, between about 7 inches and 24 inches long, etc.) and may have any appropriate outer diameter, including, but not limited to between 1 French (Fr), e.g., ⅓ mm and 34 Fr (e.g., 11.333 mm) (between 3 Fr and 30 Fr, between 4 Fr and 15 Fr, 30 Fr or less, 25 Fr or less, 22 Fr or less, 20 Fr or less, 18 Fr or less, 16 Fr or less, 15 Fr or less, 14 Fr or less, 12 Fr or less, 10 Fr or less, 9 Fr or less, 8 Fr or less, etc.).

In general, an elongate applicator tool as described herein (e.g., a high voltage, sub-microsecond elongate applicator tool) may include an elongate body with a distal end region including one or more electrodes and/or pairs of electrodes, and a proximal end including a handle and one or more controls (e.g., control elements or mechanisms) for actuating the electrodes (switching on/off) at the distal end region of the elongate applicator tool. The distal end region of the high voltage elongate applicator tool may be configured to allow manipulation of the portion of the elongate applicator tool including the one or more electrode. For example, one or more contact projections at the distal end region or tip that can be actuated to smooth the tissue near the electrodes; the vacuum may be applied automatically or manually, e.g., by a control at the proximal end.

Any of the elongate applicator tools (e.g., including catheters) described herein may include one or more electrodes of any configuration, including coil electrodes, needle electrodes, plate electrodes, bar electrodes, knife electrodes, etc. In some examples these electrodes may be variable depth needle electrodes that can penetrate tissue to a selectable variable depth. The electrodes may be on either a straight non-articulating shaft or in some examples, on an articulating shaft. Needle electrode may be actuated by a control on the proximal end and may be configured to extend out of the end region of the applicator tool.

Any of the elongate applicator tools described herein may also be configured to couple to a pulse generator for delivering high voltage, sub-nanosecond pulses into the target tissue. In particular, the elongate applicator tool (apparatuses, devices, or systems including them) may be configured to isolate the high voltage power applied by the pulse generator from the hands of the operator to prevent accidental harm to the operator. In some examples, the connection to the pulse generator may be an electrical cable that is connected to the elongate applicator tool distally from the handle. In some examples the electrical cable connection to the pulse generator may be connected to the handle or proximally to the handle. In some examples it may be beneficial to connect distally of the handle to further isolate the operator from the high voltage energy applied by the pulse generator. The electrical cable may be electrically isolated (and insulated) from the handle, and therefore the operator's hand(s), by one or more isolation elements. In some examples, in which the handle includes one or more electrical components, such as switches (including on/off switches communicating with the pulse generator), sensors or the like, may be separately powered, using an electrical connection or method that is entirely isolated and/or separate from the electrical supply of the high voltage sub-microsecond pulsed energy applied to the tissue.

Any of the apparatuses, including the elongate applicator tool and systems using them, may include one or more safety interlocking features to prevent the delivery of the high voltage, sub-microsecond pulsing until and unless the elongate applicator tool is properly deployed and in contact with a tissue, e.g., target tissue. For example, the methods and apparatuses described herein may be configured to emit one or a pattern of test pulses at very low power (e.g., low voltage) including at sub-microsecond rates to detect one or more properties of the electrical pathway including appropriate contact with a target tissue. In some examples, the apparatus may be configured to determine and detect the impedance at the one or more pairs of electrodes of the elongate applicator tool to confirm that the contact with the tissue (and the electrical pathway from the pulse generator to the tissue) are correct. Thus, these apparatuses and methods of use may include measuring an impedance of the tissue with the electrodes. In some examples, the electrodes can be used to measure the impedance of the target tissue to be treated as well as the surrounding tissue. For example, electrical energy can be applied to the target tissue at a known frequency. In a first example, the electrical energy can initially be a low-voltage pulsed energy until the electrodes are positioned appropriately against or within the target tissue. This proper positioning can be confirmed with the impedance measurement. Once the electrodes are positioned within or against the target tissue, the electrical energy can comprise high voltage, fast pulsed energy, such as sub-microsecond pulses. However, it should be understood that any type of pulsed electrical energy can be applied to the target tissue (microsecond, nanosecond, picosecond, etc.).

During treatment of the tissue, treatment may continue if certain conditions are met, but may otherwise be terminated. For example, when a change in the impedance of the target tissue exceeds an impedance threshold, treatment may stop. Thus, the detection of contact and/or treatment may be ongoing during a treatment as well as before a treatment. For example, applying electrical energy to the tissue can change the impedance of the target tissue by breaking down the tissue itself. This change can be measured, and when the change in impedance exceeds an impedance threshold that indicates the tissue breakdown, the electrodes can be moved within the tissue, or the treatment stopped. In another example, because the target tissue (e.g., tumor) may have different impedance from the surrounding tissue, a change in the impedance may occur because of the location of the elongate applicator tool and electrodes relative to the target tissue. Therefore, this change can be measured, and when the change in impedance exceeds an impedance threshold that indicates that location of the electrodes is outside the target tissue, the electrodes can be moved, or the treatment stopped. The movement of electrodes can occur either during each pulse or in between pulses, or during entire application of the electric energy.

As mentioned, any of the apparatuses described herein may be configured to include suction (e.g., vacuum) to assist in holding the electrodes to the tissue to be treated. Thus, in any of these apparatuses the electrodes may be adjacent to or surrounded by a suction or vacuum inlet. Thus, the vacuum (suction) may pull the tissue onto the electrodes and/or may maintain contact with the electrodes. This may remove air gaps between the electrodes and the tissue, which may reduce arcing and otherwise improve contact with the tissue. In any of these examples, the tip may include a sealing surface to channel the applied suction so that the seal may be maintained during treatment. In some examples the sealing surface may be formed of a soft silicon material on the tip. This soft silicone material may be formed as a cup or concavity to fit (e.g., seal) against the tissue.

In any of these examples the apparatus may include feedback to control the applied suction. For example, suction may be applied prior to the application of the energy (e.g., the high voltage, fast pulsed electrical energy). In some examples the suction may be automatically or manually applied before activating the application of the high voltage, fast pulsed electrical energy. Suction may be applied to a predetermined level to prevent damage to the tissue. Once the energy has been applied, the suction may be released, automatically or manually. In some example, positive pressure may be applied to break any seal (e.g., to release the suction) and/or to separate the tissue from the electrodes. Positive pressure may be applied by applying fluid (e.g., saline) rather than air.

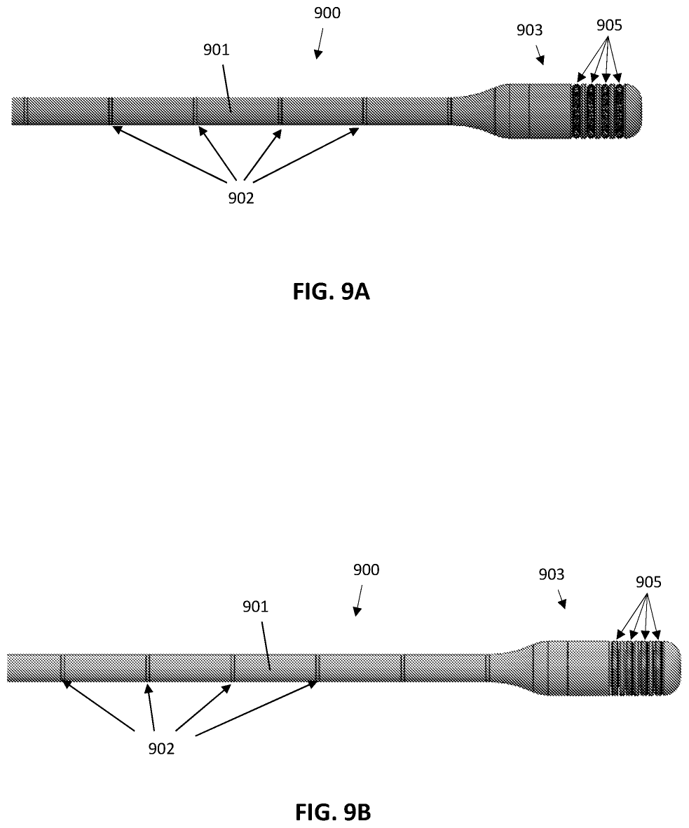

Additional examples of the apparatuses/devices of the present disclosure are described in reference to FIGS. 8-20. While, for convenience of description, these examples are discussed in reference to or for use within esophagus, it should be understood that they can be used within or for treatment of any body lumen (e.g., all other anatomical structures, passageways, tubes, lumens and tissue mentioned in this disclosure, including without limitation, otolaryngological structures, parotid glands, submucosal glandular structures, structures of the gastrointestinal tract, respiratory tract, including the trachea, pharynx, larynx, bronchi, blood vessels, uterus, fallopian tubes, etc.) Therefore, the following descriptions refer to esophagus for purposes of an example only.

FIGS. 8A-8B illustrate another example of an apparatus (e.g., an elongate applicator tool) similar to that shown in FIG. 7. In FIG. 8A, the elongate applicator apparatus is configured as a circumferential electrode, appropriate for use, for example, within an esophagus. The distal end may be used to apply pulsed energy to treat the inner circumference of the anatomical structure, such as esophagus. In FIG. 8A the apparatus 800 includes an elongate shaft 801. The shaft may be any appropriate length (e.g., for the relevant body lumen), including between 1 cm and 50 cm or more (e.g., between 10 cm and 60 cm, etc.). The shaft may be formed of a biocompatible polymeric material and may include markings 802 at regular or different intervals that may be labeled to indicate depth and/or position. A distal tip region 803 extends from the distal end of the apparatus and in this example has an outer diameter that is slightly larger than the outer diameter of the shaft. A plurality of electrodes 805 are arranged circumferentially around the distal tip region. In FIG. 8A the electrodes shown are spring electrodes, formed by a coiled wire that extends continuously around the circumference of the distal tip region, which is shown in greater detail in FIG. 8B. In general, any appropriate electrodes may be used, including (but not limited to) spring electrodes, plate electrodes, tubular electrodes, bar electrodes, or wire electrodes. In FIGS. 8A and 8B four electrodes are shown, but any appropriate number of electrodes may be used (e.g., one or more, including 2 to 50, 2 to 40, 2 to 30, 2 to 20, 2 to 10, etc.). The electrodes may be configured to be energized (for the application of pulsed electrical energy, including nanosecond pulsing) simultaneously or individually. Multiplexing individual electrodes may create a larger treatment. In any of the examples shown, the groups of electrodes may be coupled together to form anodic and cathodic electrodes or sets of electrodes to apply energy for bipolar application of energy. Alternatively these electrodes may be used in a monopolar configuration and may be used with a separate return electrode or electrodes (e.g., a grounding pad).

The apparatus shown in FIGS. 8A-8B may also include one or more openings (not visible in FIG. 8A-8B) into a vacuum port adjacent, e.g., surrounding and/or under and/or between, the electrodes. The vacuum port may couple to a vacuum channel around or between the electrodes which may help remove any air gaps between the electrodes or between the electrodes and the tissue and may also help hold the tissue in place during treatment.

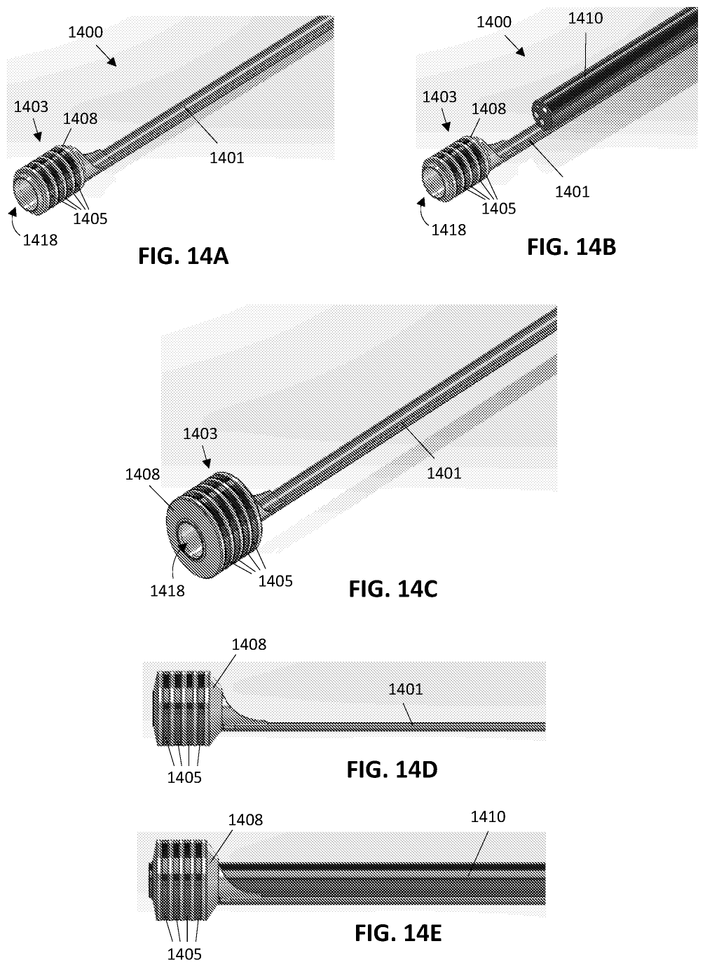

The distal tip region may be configured to be any appropriate size for circumferential treatment within a body lumen. For example, the distal tip region may have an outer diameter of between about 2 mm to 30 mm, and any appropriate electrode spacing (e.g., between about 0.5 mm to about 15 mm) to accommodate different sized lumen (e.g., different esophageal sizes, or other body lumens). As will be shown in detail in some of the other example apparatuses below (e.g., FIGS. 13A-13C, 14A-14E, 16A-16F, 18, 19A-19B and 20), the distal tip region and/or the electrodes may be configured to expand and contract in outer diameter, allowing them to snugly contact the tissue to be treated.

Any of the elongate applicator apparatuses described herein may be configured to couple to a pulse generator capable of generating pulsed (e.g. sub-microsecond, including nanosecond) electrical energy at high voltage. In FIG. 8A, the apparatus includes a high voltage connector 811 at a proximal end of the elongate shaft that is configured to make a secure mechanical and electrical connection to the pulse generator. In this example the connector is configured to couple to a handle/handpiece of the elongate applicator for connection to a pulse generator, such as an example pulse generator 100 of FIG. 1. The handle may include one or more controls for operating the applicator (including a trigger, vacuum control, inflation/expansion control, etc.); alternatively or additionally some or all of these controls may be part of the pulse generator as described in FIG. 1, above. The connector and the handle may be used to help position the electrode(s) in the proper location for treatment. The connector shown in FIG. 8A includes a mechanical securement (e.g., snap, lock, clasp, etc.) 815 as well as one or more electrical connectors 817, and a suction line connector 813.

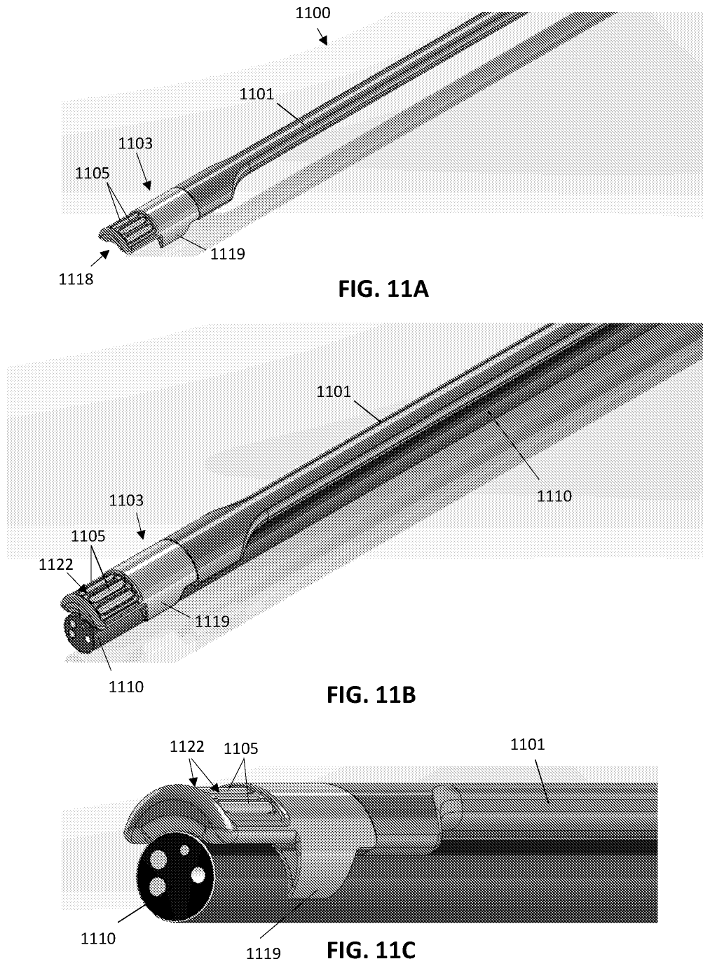

In FIGS. 8A-8B the electrodes extend around the circumference of the distal tip region and are spaced apart from each other longitudinally (e.g., in a proximal-to-distal direction along at least a portion of the distal tip region of the applicator apparatus). The electrodes may extend completely around the circumference or partially around the circumference. Any of the elongate applicator apparatuses described herein may instead have electrodes that extend longitudinally (e.g., proximal-to-distal and/or helically around the distal tip region) and are spaced apart along a circumference of the device (see, e.g., FIGS. 10A-10C, 11A-11C and 18).

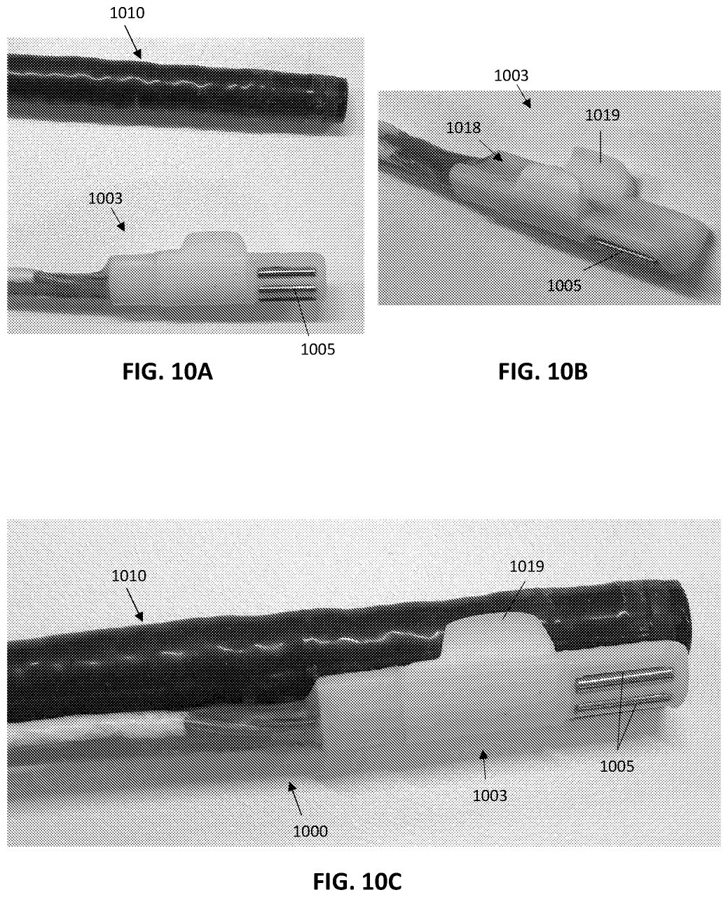

FIGS. 9A-9B schematically illustrate another example of an elongate applicator apparatus 900 similar to that shown in FIGS. 8A-8C. The distal end region, including the distal tip 903 of the apparatus is shown. In FIG. 9A the distal tip region includes a plurality of electrodes 905 arranged circumferentially around the distal tip region 903. The electrodes are shown as spring electrodes, similar to those shown in FIGS. 8A-8B. In FIG. 9B the electrodes are plate electrodes. The elongate shaft 901 shown in FIGS. 9A-9B also includes depth markers 902 that may be used to identify or confirm location/position of the distal end region during insertion into a body. The elongate shaft 901 may house the cables (e.g., power lines connecting to the electrodes) and vacuum tubing. While the examples of FIGS. 9A-B do not require the use of the endoscope or other type of a scope, in some implementations, the applicator apparatus of the present disclosure may be used in conjunction with the endoscope (e.g., bronchoscope, gastroscope, etc.).

In some examples the elongate shaft may include an open lumen extending through the distal tip region of the applicator apparatus fully or partially along the length. This lumen may be flexible and configured to allow passage of an endoscope through it and connection with the applicator apparatus. For example, FIGS. 10A-10C illustrate an example of an elongate applicator apparatus 1000 that is configured for use with an endoscope 1010. In this example the body of the distal tip region 1003 of the applicator forms a channel 1018 into which an endoscope may sit, and includes one or more coupler attachments 1019, shown in this example as a pair of wings or side projections that may extend partially around the endoscope. The channel 1018 and the attachments/wings 1019 are configured in this example to form an open channel into which an endoscope 1010 may clip or snap into. The distal tip region 1003 in FIGS. 10A-10C does not surround or extend fully around or over the endoscope, but in some examples, it may extend fully around at least a portion of an endoscope adjacent to the distal tip region 1003. Alternatively, the endoscope may be coupled to the applicator apparatus with a strap. In FIG. 10A-10C, the apparatus shows longitudinally extending electrodes 1005 that are arranged around circumference on the outer-facing surface of the distal tip region, as shown.

In use, the elongate applicator apparatus of an example of FIGS. 10A-C may be clipped onto the endoscope before, during or after insertion into the body. For example, the apparatus may be inserted into the body lumen (such as the esophagus), to treat lesions or conditions on inner sides of the body lumen. As mentioned above, the electrodes may be any appropriate electrode (e.g., springs, plates, tubes, bars, wires, etc.) and may be operated individually or may be coupled together (multiplexed). This example apparatus may also include suction ports and/or channels under, between or around the electrodes, as described above. FIG. 10A shows the distal end region of the apparatus and an example of endoscope 1010, prior to coupling the two. FIG. 10B shows a perspective view of the endoscope-facing side of the distal tip region 1003 (without the endoscope attached) including the open channel 1018 and attachments 1019. FIG. 10C shows the distal tip region 1003 coupled to the endoscope; the apparatus can be attached to the end (or side) of an endoscope by the attachment clip that is incorporated into the distal end region of the apparatus. Alternatively or additionally the two may be connected by the use of a material such as a medical tape. This may allow the visual placement of the electrodes on the target tissue (e.g., lesions) using the endoscope.

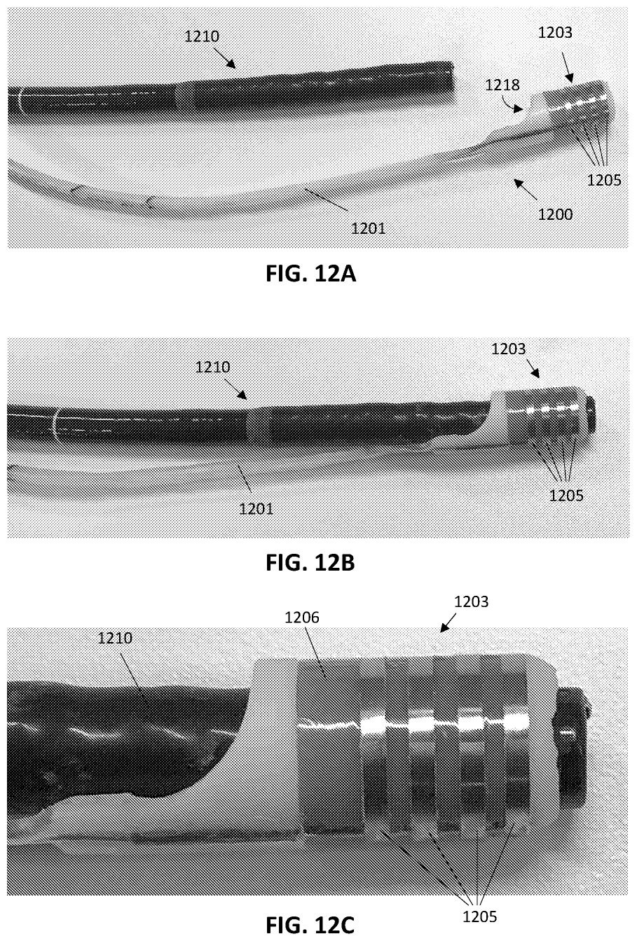

FIGS. 11A-11C schematically illustrate another example of an apparatus similar to that shown in FIGS. 10A-10C. In FIG. 11A-11C, the apparatus is also configured for side treatment and couples to an endoscope at the distal end region of the apparatus, e.g., by clipping on as shown in FIGS. 11B and 11C. This apparatus may be used to treat lesions on the inner sides of the lumen (e.g., of the esophagus).

In FIGS. 11A-11C, the apparatus includes an elongate shaft 1101. The shaft may hold or enclose wires (cables) electrically coupling the electrodes 1105 at the distal end to a connector at the proximal end (not shown) and may also include one or more suction channels (optional). The distal tip region 1103 extends from a distal end of the elongate shaft 1101, and comprises a channel 1118 (shown in this example as an open channel at the bottom, in some examples it may be closed, forming lumen) that is configured to engage an endoscope 1110. The distal tip region may include the attachments 1119 (coupler attachments) that may clip or secure the apparatus to an endoscope as shown in FIGS. 11B and 11C. The apparatus also includes a plurality of electrodes 1105 arranged along an outward-facing surface of the distal tip region. These electrodes are shown extending longitudinally and spaced circumferentially apart; alternatively they may extend circumferentially and be spaced longitudinally as shown in FIGS. 9A-B. The electrodes in this example extend in an arc of about 130 degrees, however, the arc can extend about any degree, including in a complete or nearly complete circle. In some examples the electrodes of an applicator apparatus having an open channel may extend in an arc of up to 200 degrees (e.g., 180 degrees, 160 degrees, etc.). The apparatus may also include a high voltage connector at a proximal end of the elongate shaft (not shown in FIGS. 11A-11C), that is configured to couple to a pulse generator.

The distal tip region may also include one or more openings 1122 into a vacuum port adjacent to or underneath the plurality of electrodes for applying suction, as described above.

In FIGS. 11A-11C the elongate flexible shaft 1101 has a diameter that is less than the diameter of the distal tip region and is configured to extend adjacent to the endoscope when the apparatus is coupled to an endoscope. Thus the flexible shaft may have a relatively small profile.

In any of the apparatuses described herein the distal tip region (and/or any electrodes located in the distal tip region) may be configured to expand to increase the outer diameter of the distal tip region. The electrodes may be configured to expand as the distal tip region expands. For example the distal tip region may include a balloon or other expandable/contractable member that may enlarge the effective outer diameter of the distal tip region and help secure the electrodes against the tissue.

FIG. 11C shows the apparatus coupled to an endoscope by clipping the distal tip region over the circumference of the endoscope. The endoscope may be a generic endoscope or it may be adapted for mating with the applicator apparatus. For example, the endoscope may include a mating feature (recess, snap, etc.) on the outer diameter that is configured to engage with the applicator apparatus. Complimentary mating features may be included on the distal tip region of the applicator apparatus.

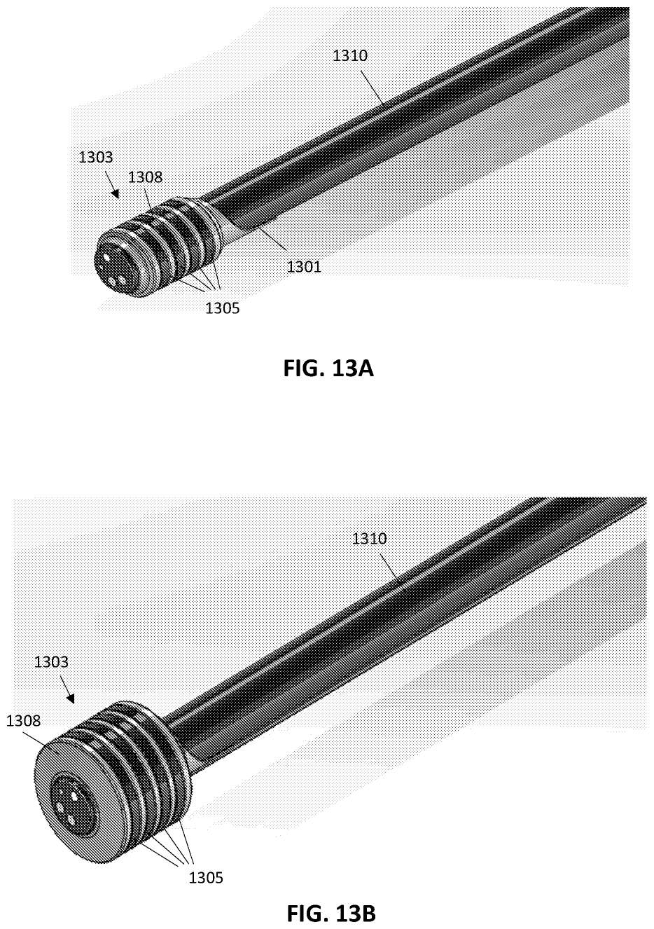

In some examples the distal tip region of the elongate applicator apparatus is configured to surround an endoscope, so that the channel is configured as a lumen extending through at least a portion of the distal tip region. For example, FIGS. 12A-12C show an example of an elongate applicator apparatus including an endoscope attachment region at the distal tip into which an endoscope may be inserted. The endoscope may be held in a fixed position (e.g., fixed longitudinal and/or rotational position) relative to the applicator apparatus or it may be free to slide distally out of the channel.

In FIGS. 12A-12C the apparatus 1200 also includes an elongate flexible shaft 1201 and the distal tip region 1203 extends distally from the shaft. The shaft may include electrical wires, suction, etc. The distal tip region 1203 includes a channel 1218 forming a lumen into which the endoscope 1210 may be inserted, as shown in FIGS. 12B and 12C. In this example the plurality of electrodes 1205 are formed in a flexible printed circuit design; the bands of electrodes are formed on an electrically insulating substrate 1206 (e.g., polyimide, or another insulating polymer). Thus, an electrode assembly can be attached to the outside of the distal end of an endoscope, as shown. Coupling the apparatus to an endoscope may allow the visual placement of the electrode on the lesions using the endoscope inside the body lumen (e.g., esophagus).

The configuration of the channel for the endoscope in these apparatuses may be similar to a rapid exchange arrangement in which the endoscope may engage just the distal tip region of the applicator apparatus. Alternatively, in some examples the elongate shaft may include one or more additional engagements for coupling to the endoscope along its length, such as clips or the like.

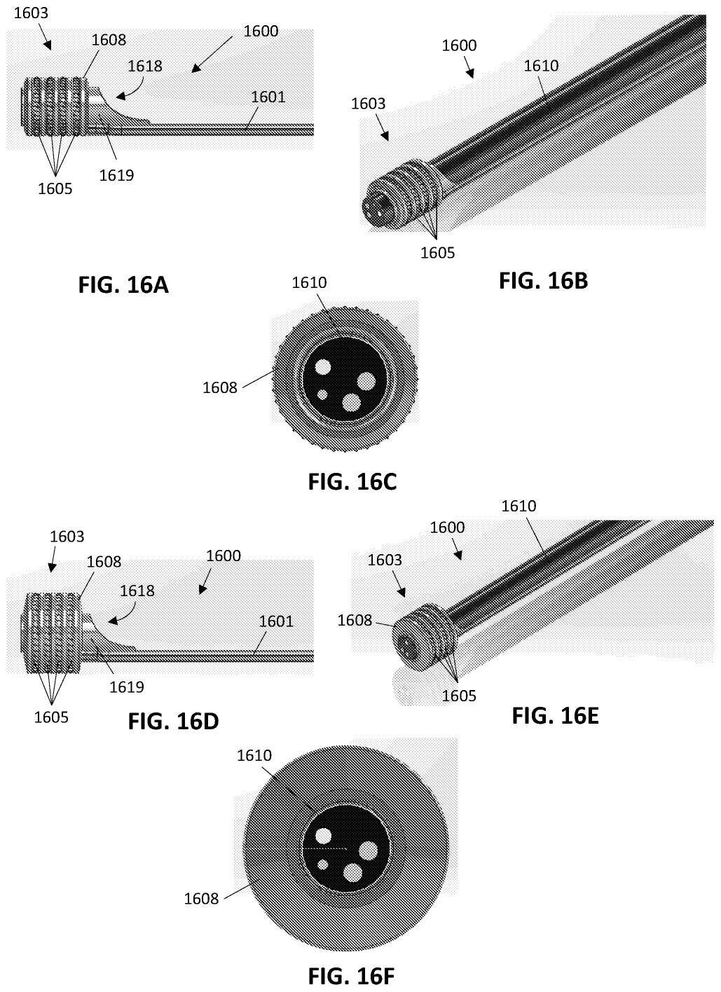

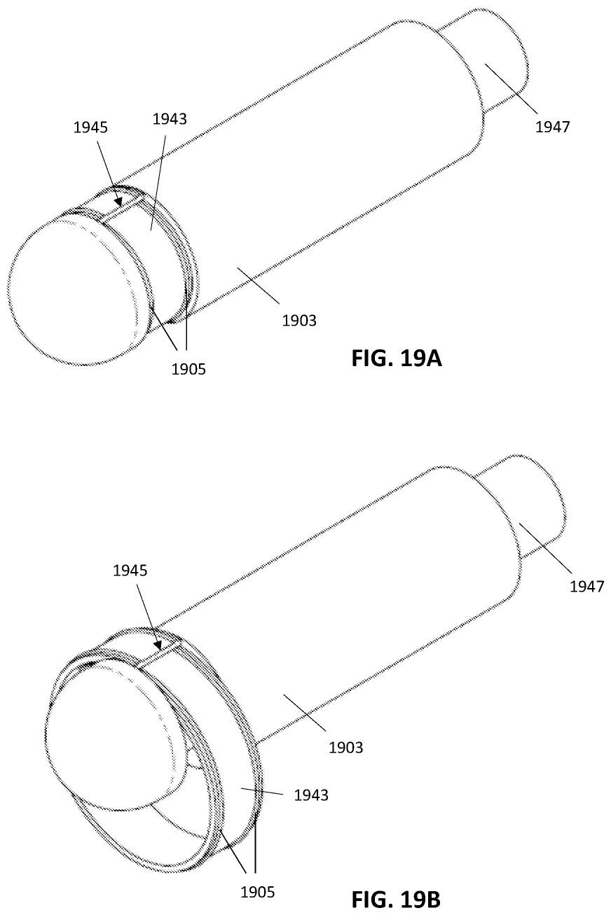

As mentioned, any of these apparatuses may include an expandable distal end region and/or expandable electrodes on the distal end region. For example, FIGS. 13A-13B show an elongate applicator apparatus having an expandable distal tip region that fits over an endoscope, as shown. In this example the apparatus includes an elongate shaft 1301 and a distal tip region 1303 that includes a channel extending longitudinally through, forming a channel for holding the endoscope 1310. The distal tip region includes a plurality of electrodes 1305 that are formed as expandable electrodes; in FIGS. 13A-13B they are shown as expandable band electrodes that are formed of a curved band or belt of conductive material that overlaps on itself and may slide over itself to expand or constrict, as described in FIGS. 15A-15D. Any appropriate expandable electrode may be used, including spring electrodes, etc. (see, e.g. FIGS. 17A-17B). The distal tip region 1303 may include an expandable outer region 1308, such as a balloon, expandable frame, expandable mesh, etc. that may be controllably expanded and/or constricted. FIG. 13A shows the distal tip region in a collapsed or retracted (constricted) configuration; FIG. 13B shows the distal tip region with the expandable outer region expanded, driving expansion of the electrodes, as shown. In FIG. 13B the expandable outer region may be a balloon or other mechanically expandable member. An expansion/contraction actuator (not shown) may be included and may extend proximally, e.g., within or parallel to the elongate shaft. The actuator may include an inflation lumen for delivering a fluid or air (e.g., saline, CO₂, etc.) to drive inflation/deflation, or a tendon to drive mechanical expansion/contraction, etc. In the example apparatus shown in FIG. 13A-13B the distal tip region may expand, for example up to 30 mm in diameter or greater, which may be particularly well suited for the esophagus. Other diameters may be used and may be matched for use in other body lumen.

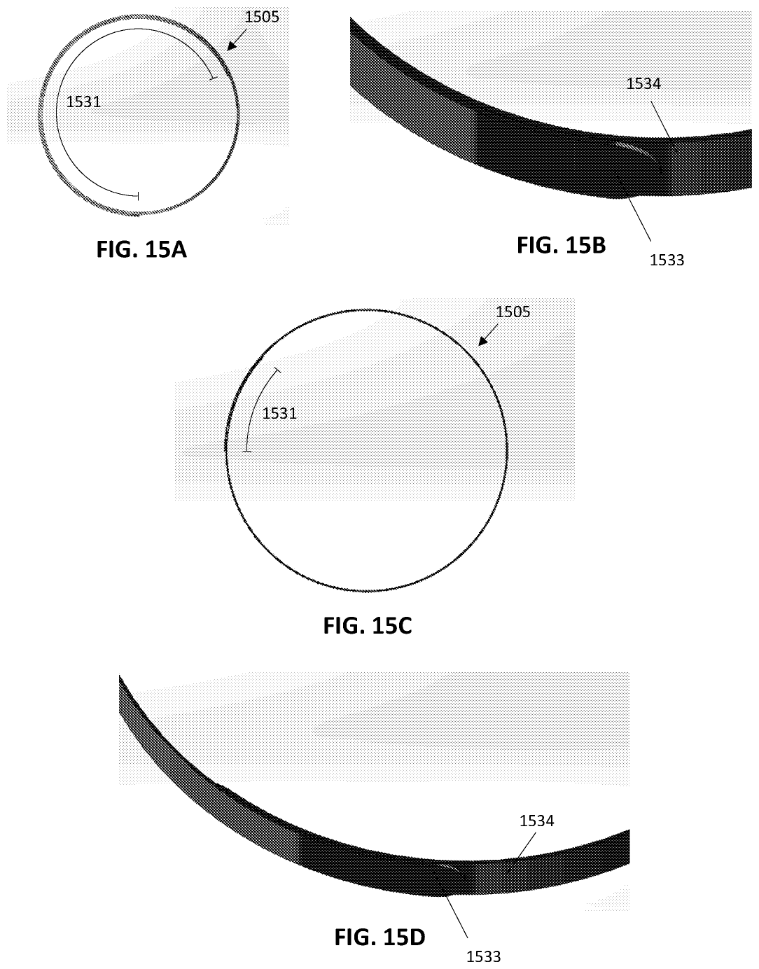

FIGS. 14A-14E illustrate the operation of an apparatus such as the one shown in FIGS. 13A-13B. In FIG. 14A the apparatus 1400 includes expandable circumferential band electrodes 1405 and a channel through the distal tip region 1403 that forms a lumen into which an endoscope 1410 may fit. This apparatus is especially useful for treating an inner circumference of a body lumen using endoscopic guidance. The apparatus shown in FIGS. 14A-14E includes an expandable outer region 1408 that is an inflatable balloon, configured as a bellows-shaped balloon with expandable band electrodes arranged circumferentially and spaced from each other longitudinally along at least a portion of the distal tip region. The electrical connectors (e.g. wires) and inflation lumen may be contained within the elongate shaft 1401. An endoscope may be coupled to the applicator apparatus by inserting the endoscope into the channel 1418 through the distal tip region 1403, as shown in FIG. 14B.

In this example, when air is applied to balloon 1408, the electrodes 1405 expand outward with the balloon, as shown in FIGS. 14C-14E, and may be held in continuous contact with inner wall of the target lumen. In use, the apparatus may be coupled with an endoscope 1410, as shown in FIG. 14.E The endoscope fits within the channel (e.g., lumen) formed by the distal tip region 1403 so that the endoscope may be used for visualization of the targeted location during placement of the active portion of the electrodes during treatment. The amount of expansion of the distal tip region may be controlled by radially expanding the expandable outer region of the distal tip region more or less based on the size of the lumen. Feedback, such as pressure feedback on the actuator/inflation lumen may be used to control the amount of expansion. Alternatively the electrical contact (e.g. impedance) with the tissue may be monitored by the apparatus to confirm sufficient contact with the tissue and may be used to assist in controlling inflation, using the expandable electrodes 1405. In some examples the endoscope may visually confirm placement and contact with the lumen.

FIGS. 15A-15D illustrate one example of a radially expandable electrode, which may be referred to herein as an overlap band electrode (or simply “band electrode”). The electrode may expand and contract radially (circumferentially) around the circumference of the distal end region of any of these apparatuses, for example, those shown in FIGS. 13A-B. For example, the band electrode may be configured as a constant force spring that expands outward as the internal balloon is inflated. The electrical connections between the electrodes may be made by means of a flex circuit that may be encased in the elongate (e.g., flexible) shaft (e.g., a cable/air supply tubing connection assembly). FIG. 15A shows an end view of the overlap band electrode. The electrode 1505 in this example is formed of a strip or strap of conductive material (e.g., metal) that is sufficiently flexible to expand and contract. The strap of conducive material loops over itself, forming an overlapping region 1531, and a first end of the strap may be on the outside 1533 while the second end of the strap is on the inside 1534 of the loop. The entire strap is electrically conductive and may therefore form a ring-shaped electrode. One end of the strap may be connected to the distal end region of the applicator apparatus (for example, to the balloon or other expansion structure), while the other end of the strap is free to allow expansion and reduction of the overlap 1531. In FIGS. 15A and 15B the ring is constricted somewhat so that the diameter of the loop formed by the electrode is small, with a larger overlapping region 1531. The diameter of the electrode may be increased by sliding the overlapping region against itself to reduce the length of the overlap region 1531, as shown in FIG. 15C.

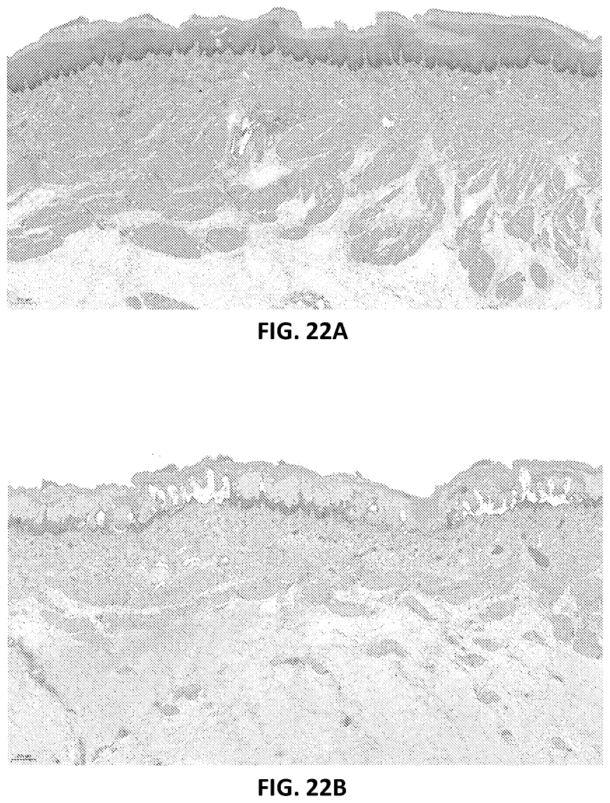

In different examples, as will be shown and described later in reference to FIGS. 19A-19B, the electrodes may instead be formed on an electrode support band that may form the mechanically expanding loop; one or more electrodes may be formed on the electrodes support, and the electrode support may be attached to the rotating actuating shaft and the distal tip region rather than the electrodes. The electrode (on the electrode support) may still slide over themselves to expand/contract.

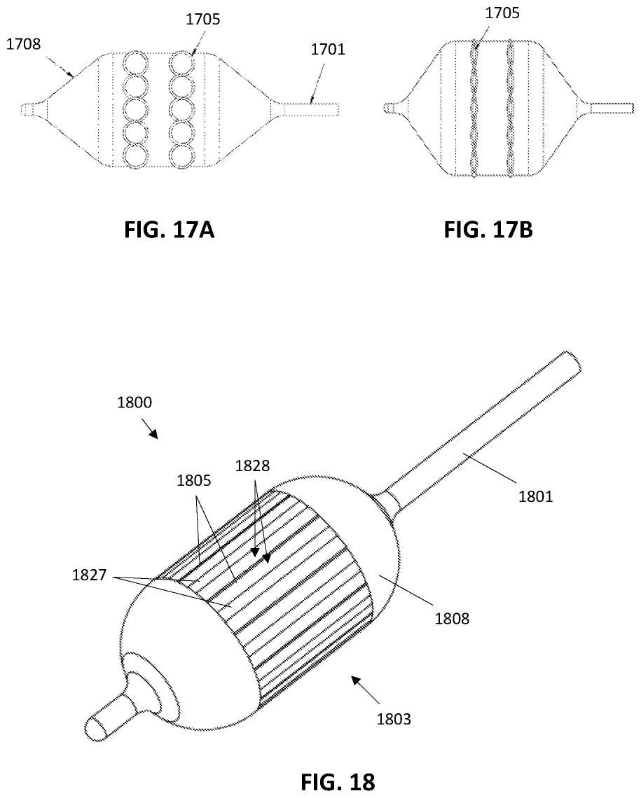

FIGS. 16A-16F illustrate another example of an applicator apparatus 1600 having an expandable set of electrodes 1605 that expand and contract with the distal tip region (shown in this example as an annular balloon that is configured to expand outwards. The expandable electrodes 1605 shown are spring electrodes formed as a ring of helically wound wire(s) forming a spring that may stretch to expand as the distal tip region expands.

The spring electrodes in this example function similar to the overlapping band electrodes shown in FIGS. 15A-15D and expandable electrodes described above; as the bellow balloon 1608 expands, the springe electrodes stretch to expand with the balloon. This is illustrated by comparing FIGS. 16A-16C, in which the distal tip region is not expanded, but is shown in a constricted/deflated configuration, with FIGS. 16D-16F, showing the distal tip region in an expanded configuration.

In FIGS. 16A-16F, the elongate applicator apparatus 1600 includes an elongate shaft 1601 that may include the conductive wires connecting the electrodes 1605 to the high voltage connector at the proximal end of the apparatus (not shown) and/or an actuator, such as an inflation lumen for expanding/contracting the expandable member 1608. The distal tip region may form a channel 1618, in this example shown as a lumen into which an endoscope 1610 may couple. In any of these examples the channel may be formed of a rigid or semi-rigid material that prevents the expandable member (e.g., balloon 1608) from compressing and reducing the lumen diameter.

In the example shown in FIGS. 16A-16F the distal tip region may also include suction ports that may apply suction under, around or between the electrodes 1605. Suction or vacuum may be used to remove airgaps around the spring electrodes. When the expandable member is inflated, the electrodes expand outward to force and hold the electrodes in continuous contact with inner wall of the of the body lumen (e.g., esophagus).

FIGS. 17A-17B illustrate another example of an expandable electrode that may be used with any of the apparatuses described. In FIGS. 17A and 17B, the electrode comprises a sinusoidal wire electrode formed by one or more wires that extend around the circumference of the distal tip region in a sinusoidal (e.g., zig-zag, helical, back-and-forth) manner that provides a spring-like ability to expand and contract. The expandable member 1708 is shown, by example, as a balloon, however, any other expandable member and corresponding method may be used, including as shown and described in reference to FIGS. 19A-C below. An elongate shaft 1701 extends proximally from the expandable member 1708. The electrode(s) 1705 may be, for example, a mesh, woven wires, or printed flexible circuits. The printed flexible circuits may have, for example, one set of “S” curves instead of two woven shown in FIGS. 17A and 17B. In some examples the electrode may be formed of one, two or more wires that form a mesh structure or any number of filaments. In FIG. 17A the electrode is shown contracted, while in FIG. 17B the electrode is expanded.

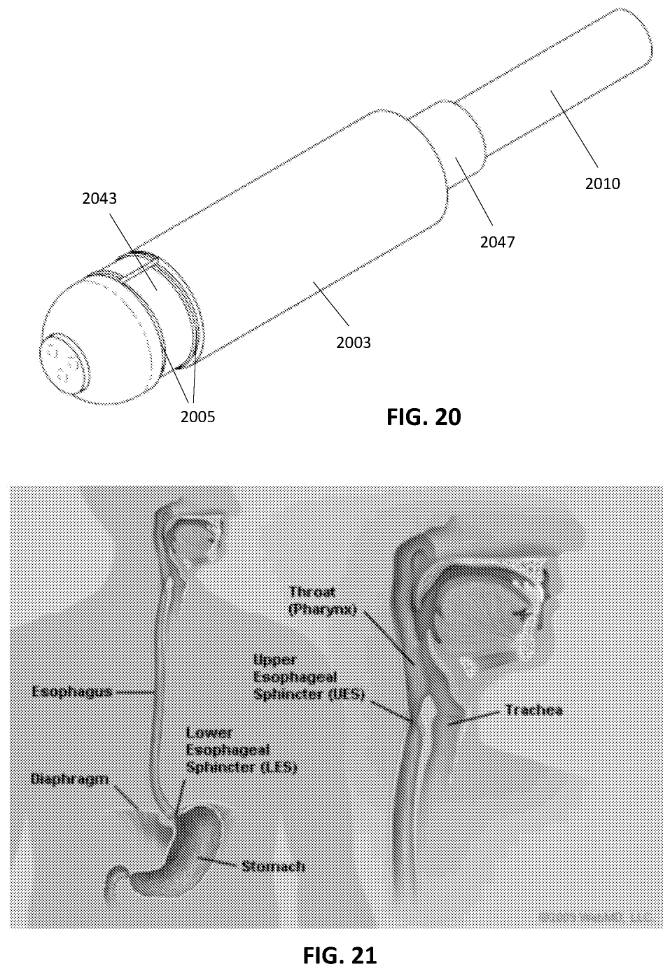

FIG. 18 shows another example of an elongate applicator apparatus having an expandable distal tip region 1803. In this example the distal tip may include a plurality of electrodes 1805 positioned circumferentially around the distal tip region 1803, extending longitudinally and separated from each other (though they may be multiplexed, as described herein). Positive electrodes may alternate from the negative electrodes. In FIG. 18 the apparatus 1800 is shown with the expandable member 1808 (configured as a balloon in this example) in an expanded configuration. An elongate shaft 1801 extends proximally and may include an inflation lumen for inflating/deflating the expandable member. The electrodes 1805 may be arranged as longitudinal conductive stripes bordered on either side, for example, by conductive spacers 1828. The conductive spacers 1828 may be any of those described in the provisional application 63/174,210 entitled “Conductive Spacer in an Electrode Assembly of an Electrical Treatment Apparatus”, which is also owned by the Applicant of the present disclosure and incorporated herein by reference in its entirety. In some embodiments, the conductive spacer may be a hydrogel, a conductive adhesive, a conductive gel, a conductive silicone, a urethane rubber, conductive thermoset, thermoplastic resins, any other biocompatible material with the desired conductivity, any semi-conductor material, or any combination thereof. In some embodiments, a conductivity of the conductive spacer may be substantially equal to ten times (10×) a conductivity of a tissue of the treatment area, however, in various examples, it may be less, equal or greater than a conductivity of a tissue of the treatment area. In some embodiments, a height of the conductive spacer may be based on a distance between the electrodes. In some embodiments, a height of the conductive spacer may be greater than twenty percent (20%) or more of a distance between the electrodes. The electrodes 1805 and conductive spacer 1828 may be further separated by additional spacers 1827 that may be insulators in some implementations or in some examples they may be further conductive spacers. It has been discovered by the inventors of the present disclosure that in some examples it may be beneficial for the electric field penetration to have two conductive spacer separated by an insulative (or less conductive) spacer. If the additional spacers 1827 are also conductive spacers, they may have conductivity that is different (e.g., lower) from the conductivity of the first conductive spacers 1828. The electrodes, as in any of these apparatuses may be multiplexed in a pattern such as alternating polarity (e.g. positive/negative). The conductive spacers described in the provisional application 63/174,210 and similar to those described in reference to FIG. 18 may be incorporated, as appropriate, in any of the applicator tools described herein in reference to various examples and Figures.

FIGS. 19A-19B illustrate an example of an apparatus in which the electrodes may expand and contract, but the distal tip region where these electrode are positioned may remain the same outer diameter. In this example the electrode(s) may be band electrode(s) and may be part of or may form a mechanically expanding ring. The electrode 1905 is formed on (or may itself form) a strip or band of material that is coupled to both an outer distal tip region of the apparatus near or at one end, and coupled at or near the other end to a rotating actuating shaft that may rotate relative to the outer distal tip region. The band of material may be referred to as an electrode support, such as electrode support 1943. The electrode support may be an electrically insulating material; the electrodes may be attached to the electrode support and the electrode support may be capable of repeated cycles of expanding and contracting without damage. The electrodes and/or electrode support may be biased to maintain the contracted configuration (FIG. 19A), or it may be biased to maintain an expanded configuration (FIG. 19B). In FIGS. 19A-19B two electrodes are formed on the electrode support; in some examples more than two electrodes may be formed (e.g., three, four, five, six, etc.). Multiple electrode supports may be used and coupled to the same actuating shaft. As described above for FIGS. 15A-15D, in some examples an electrode support is not used, but instead the electrode itself may be used to form the band.

In FIG. 19A the device is shown with the band electrodes 1905 in the constricted position, having an outer diameter that is approximately the same as the distal tip region 1903 of the apparatus. One end 1945 of the electrode support, such as the expanding ring 1943, (and therefore the band electrodes) is fixed, for example, to the outer surface or housing of the distal tip region 1903, while the other end (not shown) of the electrode support 1943 is attached to the rotatable actuating shaft 1947. Rotating the actuating shaft relative to the distal tip region may eccentrically expand and contract the electrodes as the electrode support is expanded outwards or contracted inwards. FIG. 19B shows the band electrodes 1905 expanded following rotation of the actuating shaft. As stated above, in some implementations (not shown), multiple electrode supports 1943 (or multiple bands of electrodes) may be spaced apart along the length of the distal tip region 1903. This may provide an additional benefit of better conformity to the body lumen (including to the wall of the relevant anatomical structure). For example, some or all of the spaced apart bands of electrodes (or electrode supports) may have different compliance. As a result, when expanded, they may conform differently to the wall of the body lumen providing an improved conformance of the device to the wall of the relevant anatomical structure, especially when such structure has irregular shape or changing diameter/circumference.

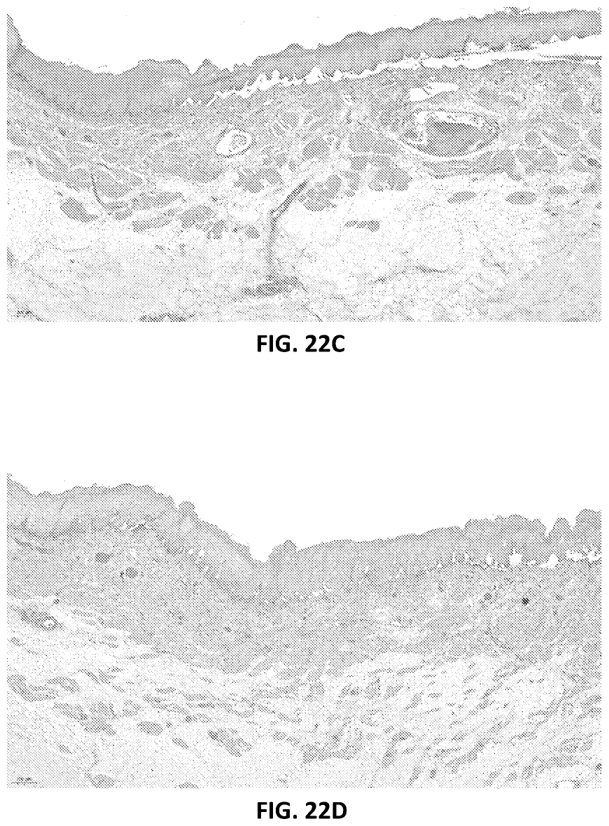

In any of these apparatuses a lumen or a channel may be included for coupling to an endoscope 2010, as shown in FIG. 20. In FIG. 20 an apparatus similar to that shown in FIGS. 19A-19B is illustrated, showing an endoscope 2010 positioned within a through channel (lumen) in the distal end region 2003 of the applicator. Two band electrodes 2005 are shown, supported on a mechanically expandable ring or loop formed by an electrode support 2043. The rotatable actuating shaft 2047 is shown as well.

### Examples

The methods and apparatuses described herein may be used to treat epithelium within a body lumen. For example, any of the apparatuses described herein may be used to ablate the epithelium of a lumen without damaging some underlying deeper layers of tissue, including for example, muscularis propria. Any of the surface (e.g. non-penetrating) electrodes described herein may be used. Nanosecond pulses may be applied as a homogenous electric field that configured to extend over the surface of the electrode.

### A. Epithelial Treatment (Including Barrett's Esophagus Treatment)

Treatment of various anatomical structures and tissue, such as esophagus, for example, can be performed with sub-microsecond electric pulses to eliminate cellular components of the mucosa or submucosa without causing damage to the muscularis externa, muscularis propria, or collagen rich connective tissues of the lamina propria. For example, muscularis externa is a large band of striated muscle surrounding the esophagus that serves structural and peristaltic functions, so it is desirable to minimize any potential impact on this layer. Therefore, sub-microsecond pulsed electric field treatment can be used to ablate the epithelium, mucosal glands, and muscularis mucosae. Due to the initiation of regulated cell death by such treatment, these cellular tissues are eliminated without an inflammatory response. Consequently, the device and methods of the present disclosure provide additional benefit of avoiding or substantially reducing generation of fibrotic tissue which often leads to stricture formation.

Thus, described herein are methods of applying energy within a lumen of the body (including but not limited to the esophagus or other otolaryngology region) to modify the tissue while preventing stricture. Stricture may be the result of scarring. In general, the methods described herein may provide nanosecond pulsing (as described herein) to ablate the mucosal epithelium to eliminate or reduce scar formation. For example, these methods and apparatuses may provide resurfacing of the mucosal epithelium or removal of tumor or dysplastic tissue while preventing stricture. In some examples regions already having stricture may be treated. One example of the application of the devices and methods of the present disclosure is for treatment of Barrett's esophagus. Barrett's esophagus greatly increases risk of esophageal adenocarcinoma. Various existing prior art devices that are used for treatment of Barrett's esophagus are limited in adoption because they often result in post-ablation stricture formation and pain. Novel devices and methods of the present disclosure, however, achieve elimination of the esophageal mucosa and submucosal glands while sparing structural collages and avoiding or substantially reducing fibrosis and stricture, as described in more detail below.

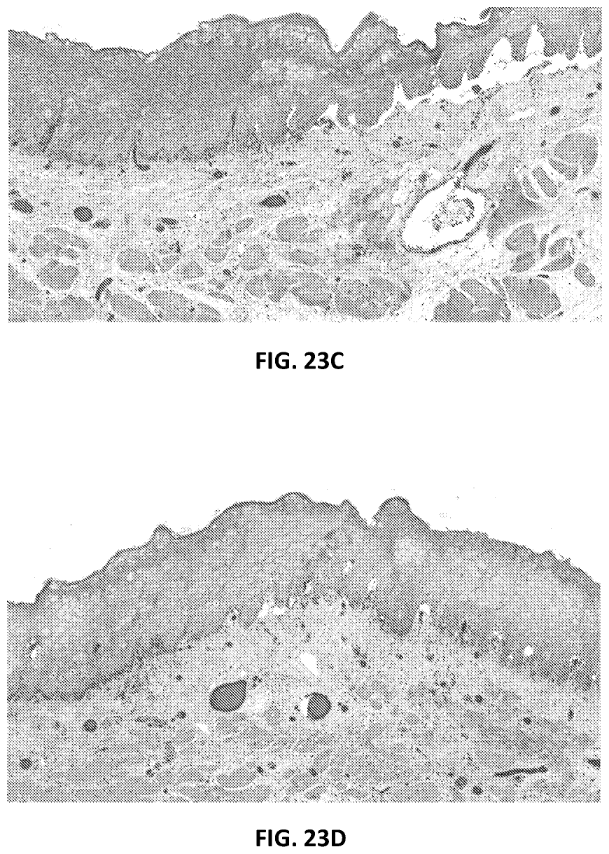

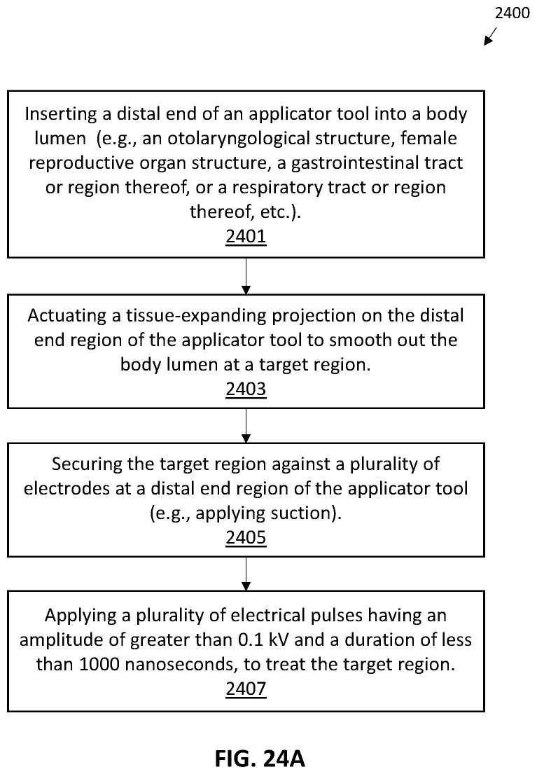

For example, FIGS. 22A-22D and 23A-23D illustrate the results of one exemplary treatment at different power levels for an esophageal region of an animal (e.g., swine). These illustrated examples of treating esophageal mucosa and treatment effect on esophageal epithelium, submucosal glandular structures and fibroses are described herein. In this example, acute ablation was applied by delivering nanosecond electrical energy against the wall of the esophageal lumen, as described above. Esophageal resurfacing was shown by 12 hours post-treatment for all samples treated, using a porcine esophagus. FIG. 22A shows an example of a control (untreated) tissue section. FIG. 22B shows a similar region of esophageal tissue 12 hours after the application of 2.3 J of nanosecond pulses. FIG. 22C shows another example, following 12 hours after treatment of this region of tissue with 5.1 J of nanosecond pulses. Finally, FIG. 22D shows a region of esophageal tissue 12 hour after treatment with 10.9 J of nanosecond field. In some treatments, energy delivered was between 1 J and 12 J with powers between 0.2 W and 5 W. In some examples, energy delivered during treatment averaged 10.85±0.29 J, with an average power of 0.56±0.05 W. All of these tissue samples (FIGS. 4A-4D) were stained with hematoxylin and eosin and captured at 4× magnification. As shown in FIG. 22A compared with each of 22B-22D, the treatment at all power levels applied resulted in near-complete ablation of the outer esophageal mucosal layer but did not significantly impact the deeper layer.

FIG. 22A shows the intact and viable epithelium from the control tissue. FIG. 22B shows ablated epithelium (after applying 2.3 J of energy), with islands of viable basal epithelium shown. FIG. 22C shows a mostly necrotic epithelium following 5.1 J of nanosecond field energy applied, showing a single layer of potentially viable basal cells. FIG. 22D shows a fully ablated epithelium with basal cells showing evidence of pyknosis following the application of 10.9 J of nanosecond pulses.

FIGS. 23A-23D show similar sections, stained with Movat's pentachrome and captured at 10× magnification. In FIGS. 23A-23D esophageal resurfacing is again visible. All of the images are taken at approximately 12 hours post-treatment for samples treated as described above. FIG. 23A shows the control tissue (untreated), while FIG. 23B shows tissue that was treated with 2.3 J of nanosecond field pulses. FIG. 23C shows tissue that was treated with 5.1 J of nanosecond field pulses and FIG. 23D shows tissue treated with 10.9 J of nanosecond field pulses.

In FIG. 23A the untreated control epithelial layer of the esophagus is similar to that of the skin. The basal layer 2301 is above epithelial cells 2303 and yellowish connective tissue 2305, which also includes the submucosa (including submucosal glands) and any underlying layer of muscle and connective tissue. FIGS. 23A-23D show acute ablation at each energy (2.3 J, 5.1 J or 10.9 J) replicated in the follow-on chronic lab. FIGS. 23A-23D show increasing amount of nonviable epithelium and provide additional evidence of pyknotic nuclei in basal epithelium in image D. Importantly, histology of the treated tissue, including those shown in FIGS. 23A-23D, showed that submucosal glands and muscularis mucosa had been eliminated with no evident damage to the muscularis propria and no change in collagen composition with increased energy, suggesting sparing of acellular structures by the applied nanosecond pulsed electric field. Esophagoscopy performed at the end of each treatment timepoint provided no evidence of stricture formation.

Therefore, as mentioned above, methods and apparatuses described herein may be particularly well suited to treat Barrett's Esophagus. Any of these methods, including methods for treatment of esophageal disorders such as Barrett's esophagus, may be effectively treated with a single circumferential ablation. This is particularly advantageous as compared to existing devices, providing a simplified single treatment as opposed to multiple steps that must be performed with existing techniques, including treatment with traditional RF devices.

For example, when treating Barrett's esophagus, the current standard of care is to treat with an expandable balloon device, to apply thermal energy, such as thermal RF treatment to ablate the Barrett's epithelium. A balloon catheter may be used to treat a full circumferential section of the esophagus, but only when there is Barrett's epithelium around all or most of the circumference of the distal esophagus. However, in many cases only a portion of the circumference of the esophagus is covered with Barrett's epithelium. For example, it is common for those with Barrett's esophagus to have “fingers” of Barrett's epithelium that extend up from the lower esophageal sphincter into the lower esophagus. Because known procedures typically employ RF thermal ablation, there is risk of fibrosis, stricture and nerve damage that can cause significant post-procedure pain. As a result, known procedures compelled to employ multiple treatment devices of different configurations in the same procedure. These treatments are performed by applying energy to only a portion of the circumference using a device of one configuration and certain size, preferably avoiding regions that do not include Barrett's epithelium; and then using a treatment device of a different configuration and/or size by applying energy to the missed portions.

Strikingly, the methods and apparatuses described herein that include applying sub-microsecond pulses of electrical energy may avoid any adverse effect on the healthy esophagus, as the applied energy may treat just some of the tissue, including cellular tissue, but may have a minimal (or no) effect on other regions. This may reduce the requirement for different sizes and configurations of applicators that are needed for the application of thermal treatments (e.g., RF treatments). Instead, the methods and apparatuses described herein may be used to apply a full circumferential treatment with the same applicator and/or may treat the entire length of the affected esophagus, treating all of the Barrett's epithelium along with the healthy epithelium without causing fibrosis, stricture or post-procedure pain. Further, the same treatment tool may be deployed multiple times, if necessary, for longer sections of affected esophagus. For example, the treatment tool may be used to treat a 2 cm section at a time. For example, if the Barrett's epithelium went up as high as 6 cm, the apparatus could be deployed three times in each of the three 2 cm regions. This provides a significant advantage over the existing methods and apparatuses. Also, the applicator tools (apparatuses) described herein may be configured to be slim enough to fit through the working channel of a standard gastroscope, so that visualization before, during and after treatment is possible without withdrawing the device. In addition, another advantage of using sub-microsecond (e.g., nanosecond) pulsed, non-thermal electrical fields in the methods of the present disclosure is that they do not cause charring of the tissue, unlike traditional thermal treatments. The traditional thermal treatments may require the user to withdraw the device, and then go back in with an endoscope and a surgical end-effector that allows the surgeon to scrape off the burned epithelium so they can visualize the tissue beneath it and ensure that all of the Barrett's epithelium has been removed. If the ablation is not complete, then they must reinsert the catheter and ablate more. A different catheter may be needed because the size of the remaining Barrett's epithelium may be smaller than the original area they treated. This makes such procedures time-consuming and tedious for the clinician, and may extend the operating time, which is not good for the patient. Because of the need to use multiple catheters, such procedures may also be more expensive. In contrast, the methods described herein may be performed in a single (or sequential using the same device) application(s) of energy to the entire affected area, with no need to scrape and/or remove and reinsert the same or different device configuration.

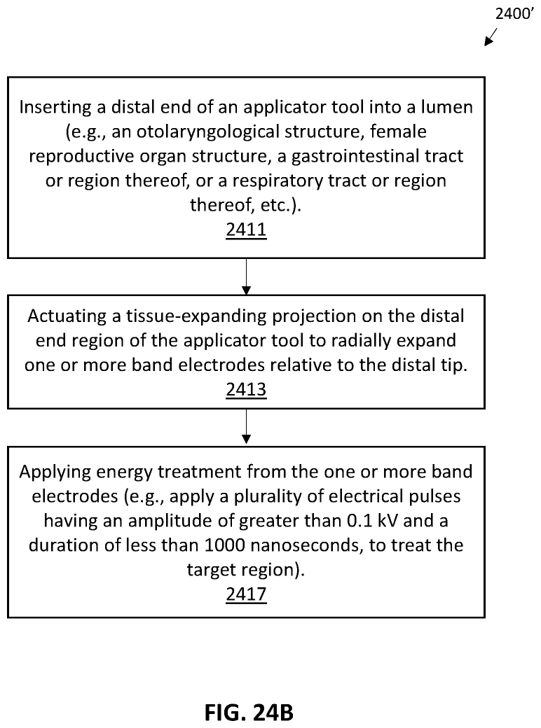

For example, FIG. 24 schematically illustrates one example of a treatment method for treating Barrett's esophagus. This method may include inserting a distal end of an applicator tool into a Barrett's esophagus of a subject, such as human or animal (step 2401). For example, Barrett's epithelium may be around only a portion of the circumference of the esophagus (e.g., “fingers” of Barrett's epithelium extending from the esophageal sphincter into the lower esophagus) or it may be a combination of the “fingers” in some portion of esophagus and full or partial circumferential Barrett's epithelium in other portions of esophagus. Once in position, which may be confirmed by visualization, an electrode assembly including one or more electrode(s), at the distal end of the applicator may be expanded to contact a full circumference of a region of the esophagus (step 2403). From the expanded configuration, the one or more (typically two or more) electrodes may be used to apply non-thermal, sub-microsecond pulsed electric field (e.g., a plurality of electrical pulses having an amplitude of equal or greater than 0.1 kV and a duration of less than 1000 nanoseconds) to treat the full (entire) circumference of the region of the esophagus (step 2405). In some cases a single (e.g., circumferential) treatment may be performed. Alternatively or additionally, when needed, multiple circumferential treatments may be applied using the same applicator tool. For example, the applicator tool may be manipulated to radially constrict the electrode assembly and move the distal end of the applicator tool longitudinally within the esophagus, so that the same steps of expanding the electrode assembly and applying non-thermal energy may be repeated (step 2507).

### B. Mucosal Resurfacing Treatment

As shown above in FIGS. 22B-22D and 23B-23D, the epithelial mucosal surface may be modified, e.g., resurfaced, by the application of nanosecond pulsed field. Any lumen having tissue that may benefit from resurfacing may be treated as described herein. For example, as discussed above, the tissue of the nasal turbinates may be resurfaced using the devices and methods described herein.

In some examples, the methods and apparatuses described herein may be used to resurface the mucosal layer of the patient's duodenum.

Duodenal resurfacing may be used or treat a patient to improve diabetes. Bypassing, excluding or altering duodenal nutrient exposure typically elicits favorable metabolic changes. Duodenal mucosal resurfacing (DMR) is one type of procedure that may be performed endoscopically to improve glycemic control in people with type 2 diabetes mellitus (T2D) irrespective of body mass index (BMI) changes. As described herein, DMR may be performed using any of the applicators included herein such as those shown and described above in FIG. 7. In this example, the applicator may be configured as a catheter that may include one or more tissue-expanding projections that may be used to provide access to the tissue of the duodenum, and completely or partially circumferential mucosal-specific ablation may be provided by nanosecond pulsed electric field. The duodenum mucosa in a region between the pylorus and ligament of Treitz region may be treated. For example, a diabetic or pre-diabetic patient may undergo DMR using these apparatuses. DMR may be feasibly and safely employed as an endoscopic procedure that elicited durable glycemic improvement in sub-optimally controlled T2D patients using oral glucose-lowering medication irrespective of weight loss.

In general, a vacuum may be used with any electrodes (e.g., coil electrodes, etc.). As described, the vacuum may be applied through multiple holes under and/or around the electrodes to maintain consistent contact with tissue and to remove any air gaps and seal around the tissue. In some examples, the tips may incorporate soft silicon material (e.g., shaped like a suction cup) for better tissue holding.

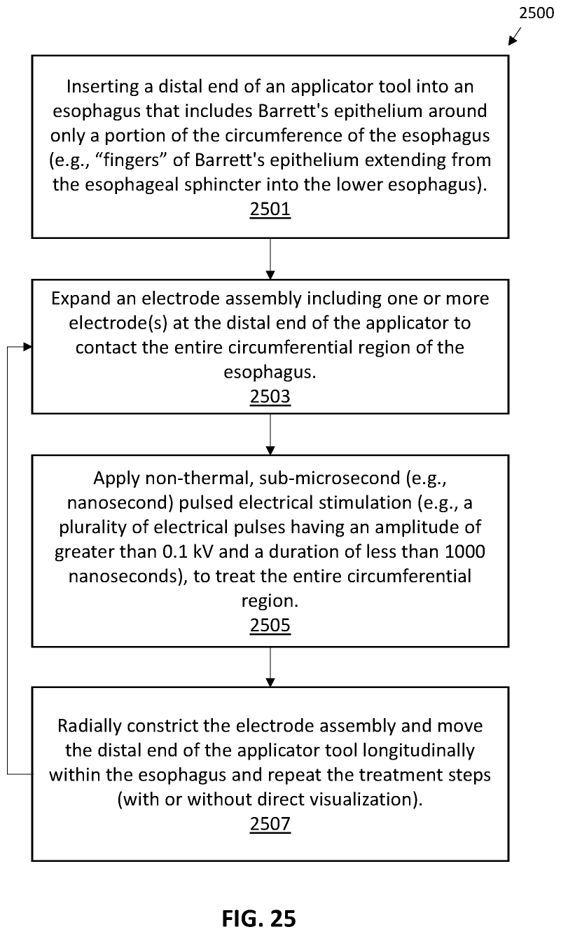

One example of a general method 2500 for treatment a lumen as describe herein in reference to various medical conditions and applications is illustrated in FIG. 25A. In this example, an applicator tool, such as one of the applicator tools described herein, may be inserted into a lumen of a subject's body (step 2501). For example, the lumen may be an otolaryngological structure, female reproductive organ structure, a gastrointestinal tract or region thereof, or a respiratory tract or region thereof, etc. The tool may include a flexible elongate body or a rigid or semi-rigid body. The tool may be steerable. In some examples, the tool may include imaging, and/or may be used with a scope for visualization. For example, the tool may include a channel for passing an imaging or visualization device.

Once in position with the lumen, so that a distal end of the applicator tool is at or near the tissue to be treated, an electrode assembly (comprising one or more electrodes) at a distal end region of the applicator tool may be deployed. For example, the electrode assembly may be deployed by radially expanding the distal end region of the applicator tool together with the electrode assembly thereon, or in some implementations the electrode assembly may be deployed by radially expanding the electrode assembly relative to the distal end region (step 2503). In any of these examples and methods described herein, optionally, this step may include smoothing (e.g., holding taut) the tissue including the target region to be treated. For example, in some implementations a tissue-expanding/contact projection(s) at or on the distal end region of the applicator tool may be actuated in order to smooth out/stretch/flatten or otherwise prepare the body lumen at a target region, as illustrated and described above. Such smoothing of tissue may be useful, for example, when treating Barrett's esophagus.

In some examples, optionally, the target region of the tissue may be secured against the electrode assembly at a distal end region of the applicator tool, for example, by applying suction (step 2505). Then pulsed electrical energy may be applied to treat the tissue, for example, by applying a plurality of electrical pulses having an amplitude of equal or greater than 0.1 kV and a duration of less than 1000 nanoseconds, to treat the target region (step 2407). The methods of the present disclosure may be used to treat epithelium of various body lumen, for example, it may be used to treat nasal turbinates, perform DMR, selectively electroporate target cells within the target tissue, ablate submucosal glands, just to name a few.

FIG. 25B illustrates another example of a method of treating a target region according to the present disclosure. The method 2500′ shown in FIG. 25B includes in step 2511 inserting a distal end of an applicator tool into a lumen (e.g., an otolaryngological structure, female reproductive organ structure, a gastrointestinal tract or region thereof, or a respiratory tract or region thereof, etc.). The method may also include deploying electrode assembly at or on a distal end region of the applicator tool, for example, by radially expanding one or more electrodes of the electrode assembly relative to the distal end region (step 2513). For example, it may not be necessary to expand the distal end region itself, so just the electrode assembly may be radially expanded. In some example, such electrode assembly may comprise one or more band electrodes. Also, it may not be necessary to smooth/flatten the target region in certain implementations. The method may further comprise applying energy treatment from the one or more electrodes of the electrode assembly to treat the target region (e.g., applying a plurality of electrical pulses having an amplitude of equal or greater than 0.1 kV and a duration of less than 1000 nanoseconds (step 2515).

In general, in any of the apparatuses described herein the surface electrode geometry may be adjusted so that the applied field from the surface electrode is more uniform along the surface of the electrode and, therefore, along the mucosal surface. For example, the surface electrodes used as part of the apparatus may include a channel or cut-out region or an insulated region within the channel region, on the back of the surface electrode facing away from the tissue surface. This channel may be a V or U shape cutout in the middle of the conductive material between 2 surface plates. In some examples a flat hydrogel layer may be present between the electrodes and the tissue; a cut-out region (e.g., a U- or V-shaped cutout) may be present in the hydrogel layer.

As mentioned above, any of the apparatuses described herein may be implemented in robotic systems that may be used to position and/or control the electrodes during a treatment. For example, a robotic system may include a movable (robotic) arm to which elongate applicator tool is coupled. Various motors and other movement devices may be incorporated to enable fine movements of an operating tip of the elongate applicator tool in multiple directions. The robotic system and/or elongate applicator tool may further include at least one image acquisition device (and preferably two for stereo vision, or more) which may be mounted in a fixed position or coupled (directly or indirectly) to a robotic arm or other controllable motion device. In some embodiments, the image acquisition device(s) may be incorporated into the elongate applicator tool.

Embodiments of the methods of the present disclosure may be implemented using computer software, firmware or hardware. Various programming languages and operating systems may be used to implement the present disclosure. The program that runs the method and system may include a separate program code including a set of instructions for performing a desired operation or may include a plurality of modules that perform such sub-operations of an operation or may be part of a single module of a larger program providing the operation. The modular construction facilitates adding, deleting, updating and/or amending the modules therein and/or features within the modules.

In some embodiments, a user may select a particular method or embodiment of this application, and the processor will run a program or algorithm associated with the selected method. In certain embodiments, various types of position sensors may be used. For example, in certain embodiment, a non-optical encoder may be used where a voltage level or polarity may be adjusted as a function of encoder signal feedback to achieve a desired angle, speed, or force.

Certain embodiments may relate to a machine-readable medium (e.g., computer readable media) or computer program products that include program instructions and/or data (including data structures) for performing various computer-implemented operations. A machine-readable medium may be used to store software and data which causes the system to perform methods of the present disclosure. The above-mentioned machine-readable medium may include any suitable medium capable of storing and transmitting information in a form accessible by processing device, for example, a computer. Some examples of the machine-readable medium include, but not limited to, magnetic disc storage such as hard disks, floppy disks, magnetic tapes. It may also include a flash memory device, optical storage, random access memory, etc. The data and program instructions may also be embodied on a carrier wave or other transport medium. Examples of program instructions include both machine code, such as produced by a compiler, and files containing higher level code that may be executed using an interpreter.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to perform or control performing of any of the steps, including but not limited to: displaying, communicating with the user, analyzing, modifying parameters (including timing, frequency, intensity, etc.), determining, alerting, or the like. In some exemplary embodiments hardware may be used in combination with software instructions to implement the present disclosure.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “mounted”, “connected”, “attached” or “coupled” to another feature or element, it can be directly mounted, connected, attached or coupled to the other feature or element or intervening features or elements may be present. In contrast, when a feature or element is referred to as being “directly mounted”, “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. As used herein, the term “and/or” includes any and all combinations of one or more of the associated listed items.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present apparatuses and methods.

The terms “comprises” and/or “comprising,” when used in this specification (including the claims), specify the presence of stated features, steps, operations, elements, and/or components, but do not preclude the presence or addition of one or more other features, steps, operations, elements, components, and/or groups thereof. Unless the context requires otherwise, “comprise”, and variations such as “comprises” and “comprising,” means various components can be co-jointly employed in the methods and articles (e.g., compositions and apparatuses including device and methods). For example, the term “comprising” will be understood to imply the inclusion of any stated elements or steps but not the exclusion of any other elements or steps.

Any of the apparatuses and methods described herein may include all or a sub-set of the components and/or steps, and these components or steps may be either non-exclusive (e.g., may include additional components and/or steps) or in some variations may be exclusive, and therefore may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the apparatuses and methods as it is set forth in the claims.

Various embodiments may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-65. (canceled)

66. An apparatus for delivering energy to a tissue, the system comprising:
an elongate applicator tool comprising:
an elongate shaft;
a distal tip region at a distal end of the elongate shaft;
a plurality of electrodes arranged along an outward-facing surface of the distal tip region;
one or more contact projections extending from either side of the plurality of electrodes and configured to be moved away from the plurality of electrodes to remove or reduce wrinkles from the tissue; and
a suction port configured to apply suction to draw the tissue against the plurality of electrodes.

67. The apparatus of claim 66, wherein the suction port comprises one or more openings adjacent to or underneath of the plurality of electrodes.

68. The apparatus of claim 66, wherein the one or more contact projections comprises a pair of arms.

69. The apparatus of claim 66, wherein the one or more contact projections are configured to rotate radially away from the plurality of electrodes.

70. The apparatus of claim 66, further comprising a control for actuating the one or more contact projections.

71. A method comprising:
placing a distal end region of an applicator tool against a wall of a body lumen, wherein the applicator tool comprises an electrode assembly at the distal end region having one or more electrodes;
moving one or more contact projections from either side of the plurality of electrodes away from the plurality of electrodes and against the wall to remove or reduce wrinkles from the wall of the body lumen;
applying suction to secure the one or more electrodes to the wall of the body lumen; and
applying energy from the one or more electrodes to treat the wall of the body lumen.

72. The method of claim 71, wherein applying energy comprises applying RF energy, microsecond pulsed energy, or sub-microsecond pulsed energy.

73. The method of claim 71, wherein energy comprises applying a plurality of electrical pulses, each pulse of the plurality of electrical pulses having an amplitude of at least 0.1 kV and a duration of less than 1000 nanoseconds.

74. The method of claim 71, wherein moving the one or more contact projections from either side of the plurality of electrodes away from the plurality of electrodes comprises rotating the one or more contact projections away from the plurality of electrodes.

75. The method of claim 71, wherein applying energy comprises applying energy to an ear, nasal cavity, mouth, tongue, turbinate, tonsils, soft palate, submucosal glands, parotid glands, esophagus, stomach, small intestine, large intestine, duodenum, pancreas and pancreatic duct, colon or fallopian tubes, uterus, urethra, cervix, trachea, pharynx, larynx, bronchi, or a mucosal epithelium of anatomical structures.

76. An elongate applicator apparatus, the apparatus comprising:
an elongate shaft;
an actuating shaft extending within or adjacent to the elongate shaft;
a distal tip region at a distal end of the elongate shaft comprising:
one or more overlap band electrodes, each comprising a strip of conductive material wrapped over itself and configured to slide over itself to radially expand to a loop having a diameter that is greater than an outer diameter of the distal tip region as the actuating shaft is rotated.

77. The apparatus of claim 76, wherein a first portion of the one or more overlap band electrodes is coupled to the actuating shaft while a second portion of the one or more overlap band electrodes is coupled to the distal tip region so that rotation of the actuating shaft drives expansion or compression of the one or more overlap band electrodes.

78. The apparatus of claim 76, wherein the distal tip region comprises a channel configured to engage a medical scope.

79. The apparatus of claim 76, wherein the one or more overlap band electrodes further comprise an electrode support onto which the strip of conductive material is coupled.

80. The apparatus of claim 76, further comprising a plurality of electrode supports, wherein each of the one or more overlap band electrodes is coupled to one of the plurality of electrode supports, further wherein each of the plurality of electrode supports is spaced from others of the plurality of electrode supports along at least a portion of a length of the distal tip region and at least one electrode support of the plurality of electrode supports having a compliance that is different from the compliance of other electrode support of the plurality of electrode supports.

81. A method, the method comprising:
inserting a distal end of an elongate applicator into a body lumen, wherein the elongate applicator comprises an overlap band electrode at a distal tip region;
rotating an actuating shaft of the elongate applicator to cause the overlap band electrode to slide over itself to radially expand to form a loop having a diameter that is greater than an outer diameter of the distal tip region; and
applying an electrical treatment to a target region on or through the body lumen.

82. The method of claim 81, wherein applying the electrical treatment comprises applying RF energy, microsecond pulsed energy, or sub-microsecond pulsed energy.

83. The method of claim 81, wherein applying the electrical treatment comprises applying a plurality of electrical pulses having an amplitude of equal or greater than 0.1 kV and a duration of less than 1000 nanoseconds.

84. The method of claim 81, wherein rotating the actuating shaft comprises rotating one end of an electrode support onto which the one or more overlap band electrodes are coupled.

85. The method of any of claim 81, wherein the target region is an ear, nasal cavity, mouth, tongue, turbinate, tonsils, soft palate, submucosal glands, parotid glands, esophagus, stomach, small intestine, large intestine, duodenum, pancreas and pancreatic duct, colon or fallopian tubes, uterus, urethra, cervix, trachea, pharynx, larynx, bronchi, or a mucosal epithelium of anatomical structures.
